(19) United States
(12) Patent Application Publication
(10) Pub. No.: US 2025/0366898 A1
(43) Pub. Date: Dec. 4, 2025

(54) SEGMENTED ELECTRODE CLAMP AND ASSOCIATED METHODS

(71) Applicant: AtriCure, Inc., Mason, OH (US)

(72) Inventors: Salvatore Privitera, Mason, OH (US); William David Dannaher, Bethel, OH (US); Kurtis Klingenberg, Cincinnati, OH (US); Matthew Monti, Cincinnati, OH (US); Michael Lambert, Cincinnati, OH (US)

(73) Assignee: AtriCure, Inc., Mason, OH (US)

(21) Appl. No.: 19/236,789

(22) Filed: Jun. 12, 2025

Related U.S. Application Data
(63) Continuation of application No. PCT/US25/10879, filed on Jan. 9, 2025.
(60) Provisional application No. 63/619,095, filed on Jan. 9, 2024.
(60) Provisional application No. 63/660,259, filed on Jun. 14, 2024.

(51) Int. Cl. A61B 18/12; A61B 18/00; A61B 18/08; A61B 90/00

(52) U.S. Cl. CPC A61B 18/1206; A61B 18/082; A61B 90/08; A61B 2018/00083; A61B 2018/00351; A61B 2018/00577; A61B 2018/00916

(57) ABSTRACT

Surgical equipment including controllers, RF generators, and surgical devices for use with ablating tissue. More specifically, surgical equipment that includes a plurality of segmented electrode pairs operatively coupled to a controller, where the controller is configured to determine, based upon impedance calculations, for each of the segmented electrode pairs whether to power the segmented electrode pairs individually or power at least some of the segmented electrode pairs as a group.

### CROSS-REFERENCE TO RELATED APPLICATIONS

The present application is a continuation of Patent Cooperation Treaty Application No. PCT/US25/10879, filed Jan. 9, 2025 and titled “SEGMENTED ELECTRODE CLAMP AND ASSOCIATED METHODS,” and claims the benefit of U.S. Provisional Patent Application No. 63/660,259, filed on Jun. 14, 2024 and titled “INTERRUPTED SPLIT ELECTRODE,” and U.S. Provisional Patent Application No. 63/619,095, filed on Jan. 9, 2024 and titled “SEGMENTED ELECTRODE CLAMP,” the disclosure of each of which is hereby incorporated by reference in its entirety.

### INTRODUCTION TO THE INVENTION

The present disclosure is directed to surgical equipment including controllers, RF generators, and surgical devices for use with ablating tissue.

The present disclosure describes, for example, an apparatus and system for ablating tissue, along with a method of use, that comprises a controller and an energy generator and a plurality of segmented electrode pairs that are adapted to be positioned in proximity to the tissue to be ablated. Each segmented electrode pair is in operative communication with the generator, and each electrode pair, when activated, generates an energy field within the tissue between the electrode pair(s). The control system (e.g., controller) is operatively associated with both the generator and the segmented electrode pairs to, in certain circumstances, continuously or alternately activate and deactivate the electrode pairs as part of carrying out a tissue ablation.

More specifically, and without limiting the foregoing, a method of tissue ablation using radio frequency (RF) electrodes as the energy source is provided in which the tissue to be ablated is contacted with a plurality of segmented electrode pairs, the electrodes of each pair being of opposite RF energy polarity so as to provide a current flux between the electrodes of each pair when activated. The electrode pairs may then be alternately activated and deactivated with RF energy to create at least one zone of primary heating in the tissue that is spaced from or substantially non-coincident with at least one zone of the highest current flux in the tissue.

In another aspect of the disclosure, a tissue ablation method is provided that comprises positioning two or more bi-directional segmented electrode pairs in spaced-apart relation in sufficient proximity to the tissue to be ablated so that, upon activation each electrode pair may create an energy field in the tissue to be ablated. The energy sources may be spaced so that the energy fields created by at least one of the activated segmented electrode pairs partially overlaps with the energy field created by a second segmented electrode pair. The segmented electrode pairs may be alternately activated and deactivated, so that a substantially constant energy field results from an area where the temporary energy fields created by at least two of the segmented electrode pairs overlap. While the energy sources may be RF energy sources, other energy sources, such as microwave, ultrasound (especially High Intensity Focused Ultrasound or HIFU), laser etc. may be used.

More specifically, a tissue ablation apparatus is disclosed herein that comprises opposed relatively moveable jaws for clamping the tissue to be ablated therebetween in the form of a clamp. A plurality of segmented electrode pairs are provided, one electrode of each pair being optionally carried on each clamp jaw and being adapted to be connected individually or at multiple pairs as a unit to the RF. A current flux may be created between the respective electrodes of each pair when supplied with energy from the RF generator. The segmented electrode pairs may be located on the jaws so that when alternately activated and deactivated by the RF generator, the electrodes create at least one flux zone of primary heating in the tissue that is spaced from at least one flux zone of the highest current flux in the tissue.

The RF energy delivered to the segmented electrode pairs may be based, at least in part, on the monitored impedance of the tissue to be ablated as it is held between the jaws of the clamp. To this end, a controller may monitor or sense voltage and/or currents associated with the segmented electrode pairs, and may be operative to calculate or derive the impedance of the tissue between electrodes on opposing sides of the tissue. The ablation may continue until the calculated impedance indicates that the lesion or ablation line is transmural (or fully through the tissue thickness) or until the impedance meets a predetermined threshold.

It is a first aspect of the present disclosure to provide an ablation system comprising: (i) a controller; (ii) an energy generator operatively coupled to the controller; and, (iii) a surgical device including a plurality of segmented electrode pairs, the plurality of segmented electrode pairs being operatively coupled to the controller but electrically isolated from one another, where the controller is configured to individually power at least a portion of or all of the plurality of segmented electrode pairs and/or to power at least a portion of or all of the plurality of segmented electrode pairs as a group.

In a more detailed embodiment of the first aspect, the surgical device comprises a surgical clamp that includes a first jaw and a second jaw, where at least one of the first jaw and the second jaw is repositionable, and the plurality of segmented electrode pairs are distributed across the first jaw and the second jaw. In yet another more detailed embodiment, the plurality of segmented electrode pairs includes a first segmented electrode pair, a second segmented electrode pair, a third segmented electrode pair, and a fourth segmented electrode pair, the first jaw includes a first electrode of each of the first segmented electrode pair, the second segmented electrode pair, the third segmented electrode pair, and the fourth segmented electrode pair, and the second jaw includes a first electrode of each of the first segmented electrode pair, the second segmented electrode pair, the third segmented electrode pair, and the fourth segmented electrode pair. In a further detailed embodiment, the controller includes a visual display including visual indicium representing each of the plurality of segmented electrode pairs, and the visual display is updated to reflect progress of an ablation sequence using the plurality of segmented electrode pairs. In still a further detailed embodiment, the visual indicium includes numerical representations regarding the progress of the ablation sequence. In a more detailed embodiment, the visual indicium includes colored representations regarding the progress of the ablation sequence. In a more detailed embodiment, the colored representations change as a function of nearing completion of the ablation sequence. In another more detailed embodiment, the colored representations include a first shape denoting a first of the plurality of segmented electrode pairs is being powered individually, and a second shape denoting at least a second of the plurality of segmented electrodes is not being powered individually. In yet another more detailed embodiment, at least two of the electrodes of the first jaw are oriented in parallel to one another. In still another more detailed embodiment, a first of the electrodes of the first jaw is oriented parallel to second and third electrodes of the first jaw, and the first electrode overlaps both the second and third electrodes in a direction normal to the parallel orientation.

It is a second aspect of the present disclosure to provide a method of carrying out an ablation process using a plurality of segmented electrode pairs, the method comprising: (i) sandwiching tissue between a first segmented electrode pair, a second segmented electrode pair, a third segmented electrode pair, and a fourth segmented electrode pair, where a first electrode of each segmented electrode pair is on a first side of the tissue, and where a second electrode of each segmented electrode pair is on a second, opposite side of the tissue; and, (ii) individually and concurrently powering each of the first segmented electrode pair, the second segmented electrode pair, the third segmented electrode pair, and the fourth segmented electrode pair without regard to power delivered to the other segmented electrode pairs.

In a more detailed embodiment of the second aspect, the method further includes determining impedance of the tissue sandwiched between the first segmented electrode pair, the second segmented electrode pair, the third segmented electrode pair, and the fourth segmented electrode pair to generate four separate tissue impedances, where each of the four separate tissue impedances are below a first threshold impedance. In yet another more detailed embodiment, the method further includes sandwiching tissue between a fifth segmented electrode pair and a sixth electrode pair, where a first electrode of the fifth and sixth electrode pairs is on the first side of the tissue, and where a second electrode of the fifth and sixth electrode pairs is on the second, opposite side of the tissue, and concurrently powering each of the fifth segmented electrode pair and the sixth segmented electrode pair by equally distributing an ablation energy from an energy generator. In a further detailed embodiment, the method further includes determining impedance of the tissue sandwiched between the fifth segmented electrode pair and the sixth segmented electrode pair, to generate fifth and sixth separate tissue impedances, where at least one of the fifth and sixth separate tissue impedances is above a first threshold impedance, and where the fifth and sixth segmented electrode pairs are operated as a single unit. In still a further detailed embodiment, the method further includes determining impedance of the tissue sandwiched between the fifth segmented electrode pair and the sixth segmented electrode pair after concurrently powering the fifth and sixth segmented electrode pairs, and discontinuing power to both the fifth and sixth segmented electrode pairs when the determined tissue impedance is above a threshold ablation metric. In a more detailed embodiment, the method further includes determining impedance of the tissue sandwiched between the first segmented electrode pair, the second segmented electrode pair, the third segmented electrode pair, and the fourth segmented electrode pair to generate four separate tissue impedances after concurrently powering the first, second, third, and fourth segmented electrode pairs, where at least one of four separate tissue impedances corresponding to the first segmented electrode pair is above a threshold ablation metric, and revising the individually and currently powering the first and second segmented electrode pairs to power the first and second segmented electrode pairs as a single group so the same power is distributed equally across the first and second segmented electrode pairs. In a more detailed embodiment, the method further includes determining impedance of the tissue sandwiched between the first segmented electrode pair, the second segmented electrode pair, the third segmented electrode pair, and the fourth segmented electrode pair to generate four separate tissue impedances after concurrently powering the first, second, third, and fourth segmented electrode pairs, where at least one of four separate tissue impedances is above a threshold ablation metric, and revising the individually and concurrently powering of those of the first, second, third, and fourth segmented electrode pairs when a tissue impedance across one or more of the first, second, third, and fourth segmented electrode pairs is above the threshold ablation metric and, instead, group powering at least two of the first, second, third, and fourth segmented electrode pairs.

It is a third aspect of the present disclosure to provide at least one of an ablation controller and an energy generator comprising: (i) an electrical connection configured to engage and establish electrical communication with a surgical device; and, (ii) a processor programmed to receive inputs from the surgical device to determine tissue impedance at a plurality of tissue locations, the processor also programmed to use the determined tissue impedance to configure whether power will be individually routed to the surgical device via a first channel or whether power will be distributed equally to the surgical device across multiple channels.

In a more detailed embodiment of the third aspect, the processor is programmed to generate instructions for a visual display to display indicia reflecting how may segmented electrode pairs the surgical device includes. In yet another more detailed embodiment, the processor is programmed to generate instructions for a visual display to display indicia for each of a plurality of segmented electrode pairs comprising the surgical device, and where the processor is programmed to update the instructions as based upon updated tissue impedance determinations.

It is a fourth aspect of the present disclosure to provide an ablation system comprising: (i) an energy generator; and, (ii) a surgical device including a plurality of segmented electrode pairs, the plurality of segmented electrode pairs being electrically isolated from one another but operatively coupled to the energy generator, where the energy generator is configured to individually power at least a portion of or all of the plurality of segmented electrode pairs and/or to power all of or at least a portion of the plurality of segmented electrode pairs as a group dependent upon tissue impedance.

In a more detailed embodiment of the fourth aspect, the surgical device comprises a surgical clamp that includes a first jaw and a second jaw, where at least one of the first jaw and the second jaw is repositionable, and the plurality of segmented electrode pairs are distributed across the first jaw and the second jaw. In yet another more detailed embodiment, the plurality of segmented electrode pairs includes a first segmented electrode pair, a second segmented electrode pair, a third segmented electrode pair, and a fourth segmented electrode pair, the first jaw includes a first electrode of each of the first segmented electrode pair, the second segmented electrode pair, the third segmented electrode pair, and the fourth segmented electrode pair, and the second jaw includes a first electrode of each of the first segmented electrode pair, the second segmented electrode pair, the third segmented electrode pair, and the fourth segmented electrode pair. In a further detailed embodiment, the energy generator includes a visual display including visual indicium representing each of the plurality of segmented electrode pairs, and the visual display is updated to reflect progress of an ablation sequence using the plurality of segmented electrode pairs. In still a further detailed embodiment, the visual indicium includes numerical representations regarding the progress of the ablation sequence. In a more detailed embodiment, the visual indicium includes colored representations regarding the progress of the ablation sequence. In a more detailed embodiment, the colored representations change as a function of nearing completion of the ablation sequence. In another more detailed embodiment, the colored representations include a first shape denoting a first of the plurality of segmented electrode pairs is being powered individually, and a second shape denoting at least a second of the plurality of segmented electrodes is not being powered individually. In yet another more detailed embodiment, at least two of the electrodes of the first jaw are oriented in parallel to one another. In still another more detailed embodiment, a first of the electrodes of the first jaw is oriented parallel to second and third electrodes of the first jaw, and the first electrode overlaps both the second and third electrodes in a direction normal to the parallel orientation.

### BRIEF DESCRIPTION OF THE DRAWINGS

FIG. 1 is a perspective view of a system according to the present disclosure including a control and RF energy generator and a cardiac ablation clamp having opposed jaws.

FIG. 2 is an enlarged fragmentary perspective view of the opposed jaws of the ablation clamp shown in FIG. 1.

FIG. 3 is a plan view of a first jaw of the ablation clamp of FIG. 1.

FIG. 4 is an enlarged cross-sectional view of the opposed jaws of the RF ablation clamp of FIG. 1 taken along line 4-4 of FIG. 3.

FIG. 5 is a plan view of a second jaw the ablation clamp of FIG. 1.

FIG. 6 is a perspective view of an example surgical clamp.

FIG. 7 is a detailed perspective view of an example end effector of the surgical clamp of FIG. 6.

FIG. 8 is a side view of the example end effector of FIG. 7 with the jaws in an open position.

FIG. 9 is a side view of the example end effector of FIG. 7 with the jaws in an intermediate position.

FIG. 10 is a side view of the example end effector of FIG. 7 with the jaws a closed position.

FIGS. 11 and 12 are perspective cutaway views of an example articulating mechanism of the clamp of FIG. 6.

FIG. 13 is an exploded perspective view of the example end effector of FIG. 7 including an example articulating mechanism.

FIGS. 14 and 15 are detailed internal perspective views of example head shell portions of the surgical clamp of FIG. 6.

FIG. 16 is an internal side view of an example handle of the surgical clamp of FIG. 6.

FIG. 17 is a detailed view of the inwardly facing (e.g., tissue-clamping) surface of an example first jaw of the surgical clamp of FIG. 6.

FIG. 18 is a detailed view of the inwardly facing (e.g., tissue-clamping) surface of an example second jaw of the surgical clamp of FIG. 6.

FIG. 19 is a magnified view of a portion of the example first jaw of FIG. 17.

FIG. 20 is a cross-sectional view of the example first jaw of FIG. 17 taken along the arrowed lines.

FIG. 21 is a perspective view of a second exemplary system according to the present disclosure including a controller and an RF energy generator and two exemplary cardiac ablation clamps having opposed jaws.

FIGS. 22A-22D are a series of exemplary visual indicators that may be displayed on a visual display in accordance with the instant disclosure.

FIGS. 23A-23C are a series of exemplary visual indicators that may be displayed, in addition to a visual reproduction of an end effector of a surgical clamp, on a visual display in accordance with the instant disclosure.

FIG. 24 are exemplary visual indicators that may be displayed on a visual display in accordance with the instant disclosure.

FIGS. 25A and 25B are a series of exemplary visual indicators that may be displayed on a visual display in accordance with the instant disclosure.

FIG. 26 is a simplified posterior perspective view of a heart showing example operations using guides and/or clamps in accordance with the instant disclosure.

FIG. 27 is a simplified superior perspective view of a heart showing an example clamp being used in accordance with the instant disclosure.

FIG. 27A is a graphical representation that may be associated with a controller display of an exemplary controller depicting which segmented electrode pairs will be driven individually and those that will be driven as a group.

FIG. 27B is a graphical representation that may be associated with a controller display of an exemplary controller depicting segmented electrode pairs surpassing a first threshold, and others with a different color or shade that have not yet met a first threshold.

FIG. 27C is a graphical representation that may be associated with a controller display of an exemplary controller depicting segmented electrode pairs surpassing a second threshold, and others with a different color or shade that have surpassed the first threshold, but not the second threshold.

FIG. 27D is a graphical representation that may be associated with a controller display of an exemplary controller depicting segmented electrode pairs surpassing a final threshold and are no longer powered, and others with a different color or shade that have surpassed the second threshold, but not the final threshold.

FIG. 27E is a graphical representation that may be associated with a controller display of an exemplary controller depicting all segmented electrode pairs surpassing a final threshold and are no longer powered.

FIG. 28 is an elevated perspective view of a jaw precursor for an electrosurgical instrument that includes rows of electrode contacts.

FIG. 29 is a top view of the jaw precursor of FIG. 28.

FIG. 30 is a side view of the jaw precursor of FIG. 28.

FIG. 31 is an elevated perspective view of a series of electrodes that may be incorporated into the jaw precursor of FIG. 28.

FIG. 32 is a top view of the series of electrodes of FIG. 31.

FIG. 33 is a profile view of the series of electrodes of FIG. 31.

### DETAILED DESCRIPTION

The exemplary embodiments of the present disclosure are described and illustrated below to encompass surgical equipment including controllers, RF generators, and surgical devices for use with ablating tissue. Of course, it will be apparent to those of ordinary skill in the art that the embodiments discussed below are exemplary in nature and may be reconfigured without departing from the scope and spirit of the present invention. However, for clarity and precision, the exemplary embodiments as discussed below may include optional steps, methods, and features that one of ordinary skill should recognize as not being a requisite to fall within the scope of the present invention.

As described above, radio frequency (RF) energy may be used in electrosurgical systems for heating, coagulation, or ablating tissue. Bipolar electrosurgical instruments apply energy between a pair of electrodes in direct contact with the tissue to be ablated and, in accordance with the instant disclosure, can provide more precise control of the extent of tissue ablation than via monopolar ablation.

In accordance with one aspect of the present disclosure, FIG. 1 illustrates an example of a tissue ablation system in the form of a bipolar electrosurgical system 10 having an electrosurgical clamp 12 coupled to an energy generator, e.g., an RF generator 14. Electrosurgical clamp 12 may include a handle 16, an elongated longitudinal shaft 17 extending therefrom, and an end effector 18 for clamping and heating tissue therebetween. Although illustrated as a clamping device particularly suited for open procedures where the ablation site is directly viewable by the surgeon, the present disclosure is amenable for use with minimally invasive procedures, such as intercostal or subxiphoid approaches to cardiac tissue targeted for ablation.

The illustrated end effector 18 has first and second opposed jaws 20, 22 for clamping tissue therebetween, henceforth referred to for convenience as proximal jaw 20 and distal jaw 22. The proximal and distal jaws 20, 22 are shown spaced apart for the reception of tissue therebetween, but at least one of the proximal and distal jaws 20, 22 respectively could be movable to clamp tissue therebetween. To this end, proximal and distal jaws 20, 22 may be operably coupled to a closure trigger 24 extending proximally from the handle 16 so that one-hand distal movement of closure trigger 24 brings the proximal and distal jaws 20, 22 together. Likewise, proximal movement of closure trigger 24 moves the proximal and distal jaws 20, 22 apart. The proximal and distal jaws 20, 22 are shown extending at an angle from the shaft 17, but can be at any angle with respect to the shaft 17. The present disclosure is not limited to the particular mechanism for moving the jaw(s) and an example of such a mechanism may be found in U.S. Pat. No. 6,923,806 and U.S. Pat. No. 7,291,161, for example, which are incorporated by reference.

The present disclosure also encompasses methods, apparatuses, and systems for ablating tissue that includes an exemplary method of positioning two or more bidirectional ablation energy sources spaced apart from one another, but in sufficient proximity to the tissue to be ablated so that, upon activation, each energy source creates an energy field in the tissue to be ablated. The energy sources may be spaced so that the energy fields created by at least one of the activated sources partially overlaps with the energy field created by one or more of the other activated energy sources. The energy sources may be alternately activated and deactivated, so that a substantially constant energy field results where the energy fields created by at least two of the energy sources overlap. While the energy sources are discussed in the context of RF energy sources, other energy sources, such as microwave, may be used.

To that end, and in keeping with one aspect of the present disclosure that employs RF energy, two or more pairs of opposed electrodes may be located in proximal and distal jaws 20, 22 of the clamp 12. Some or all of the electrodes may be operably coupled to the RF generator 14 by a cable 26. A controller (optionally incorporated into the RF generator 14 or an RF generator incorporated into a controller, or a controller or RF generator individually) may be utilized for providing RF energy.

As will be discussed in more detail hereafter, the RF energy delivered to the electrode pairs may be based, at least in part, upon impedance assessments local to one or more of the electrode pairs when the electrode pairs are interposed by tissue to be ablated. To this end, the controller may monitor or sense voltage and/or currents across electrodes, calculating or deriving the impedance of the tissue between the electrodes. As will be discussed in more detail hereafter, the impedance assessments may include an initial impedance assessment before the ablation commences and may include impedance assessments that occur in real-time as the ablation commences and may further occur even after the ablation is completed. By way of example, the ablation may commence and continue until the calculated impedance reaches or exceeds a predetermined impedance value indicative of a lesion or ablation line that is transmural (or fully through the tissue thickness). It should also be understood, however, that the predetermined impedance value may be less than that required to result in a lesion or ablation line that is transmural.

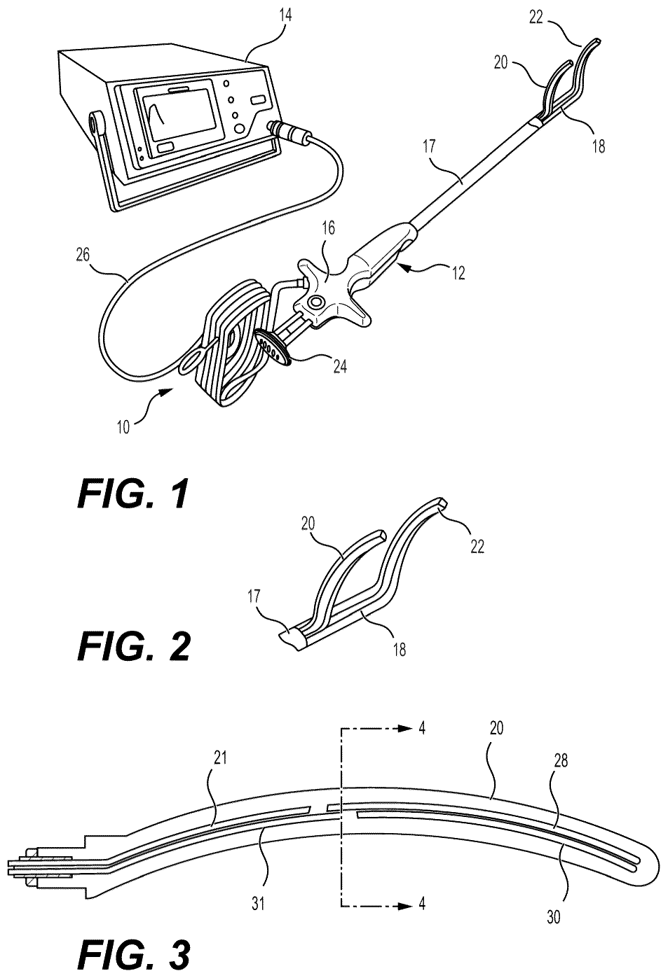

Turning to FIGS. 1-5, the electrosurgical clamp 12 may include four or more pairs of opposed electrodes. The first jaw 20 may include a first electrode 21, a second electrode 31, a third electrode 28, and a fourth electrode 30 seated within an insulator 32 that extends along the length and across the width of the jaw. Similarly, the second jaw 22 may include a corresponding first electrode 37, a corresponding second electrode 39, a corresponding third electrode 36, and a corresponding fourth electrode 38 also seated within an insulator 32. Though not required, the electrodes 21, 28, 30, 31, 36, 37, 38, 39 may terminate at different longitudinal locations along each jaw 20, 22. For example, the second electrode 31 may extend longitudinally beyond a terminal end of the first electrode 21 so that at least a portion of the second electrode is laterally across from (laterally overlaps) a portion of the third electrode 28. Likewise, the third electrode 28 may extend longitudinally beyond a terminal end of the fourth electrode 30 so that at least a portion of the third electrode is laterally across from a portion of the second electrode 31. Conversely, the electrodes 31, 31, 28, 30 may have terminal ends that approximate one another in a lateral direction. In exemplary form, the electrodes 21, 31, 28, 30 may be centered laterally on the insulator 32 about the medial plane of the jaw 20 and spaced apart a distance 34 of from about 0.7 mm to about 4.0 mm. It should be noted, however, that electrode spacings smaller than 0.7 mm and larger than 4.0 mm may be utilized. As the spacing of the electrodes 21, 31, 28, 30 is increased, the insulator 32 surface may become more convex to achieve higher pressure on the tissue between the electrodes. With four electrodes 21, 31, 28, 30, for example, the crown radius of the insulator 32 may be about 4.5 mm, and its face width may be about 5.0 mm. These dimensions are illustrative only, and other dimensions may be used without departing from the present disclosure.

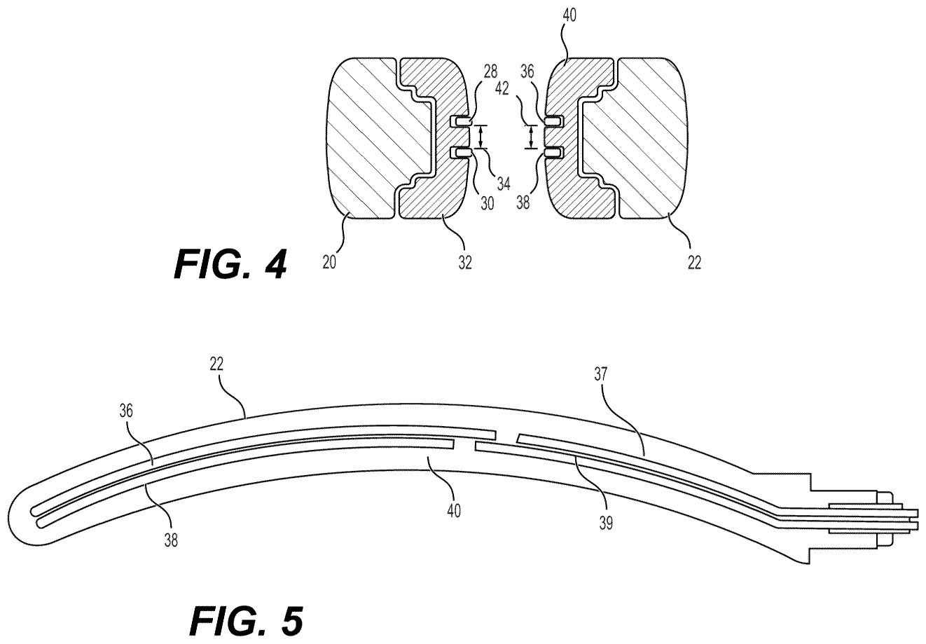

Referring to FIGS. 4 and 5, the distal jaw 22 may be configured similarly to the proximal jaw 20 and have a first corresponding electrode 37 directly opposite to the first electrode 21, a second corresponding electrode 39 directly opposite the second electrode 31, a third corresponding electrode 36 directly opposite to the third electrode 28, and a fourth corresponding electrode 38 directly opposite the fourth electrode 30, where the electrodes are seated within an insulator 32. Though not required, the electrodes 36-39 may terminate at different longitudinal locations along the second jaw 22. For example, the second corresponding electrode 39 may extend longitudinally beyond a terminal end of the first corresponding electrode 37 so that at least a portion of the second corresponding electrode is laterally across from (laterally overlaps) a portion of the third corresponding electrode 36. Likewise, the third corresponding electrode 22 may extend longitudinally beyond a terminal end of the fourth corresponding electrode 38 so that at least a portion of the third corresponding electrode is laterally across from a portion of the second corresponding electrode 39. Conversely, the electrodes 36-39 may have terminal ends that approximate one another in a lateral direction. In exemplary form, the electrodes 36-39 may be centered laterally on the insulator 32 about the medial plane of the jaw 22 and spaced apart a distance 34 of from about 0.7 mm to about 4.0 mm. It should be noted, however, that electrode spacings smaller than 0.7 mm and larger than 4.0 mm may be utilized. As the spacing of the electrodes 36-39 is increased, the insulator 32 surface may become more convex to achieve higher pressure on the tissue between the electrodes. With four electrodes 36-39, for example, the crown radius of the insulator 32 may be about 4.5 mm, and its face width may be about 5.0 mm. These dimensions are illustrative only, and other dimensions may be used without departing from the present disclosure.

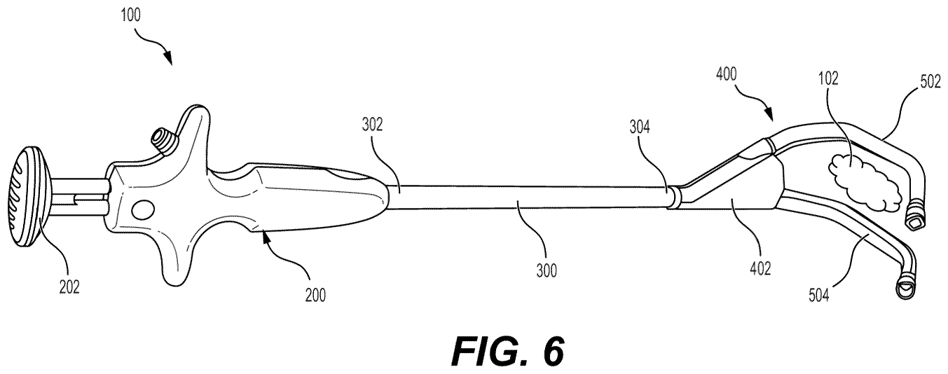

Referencing FIG. 6, a further exemplary electrosurgical instrument 100 in accordance with the instant disclosure may comprise a surgical clamp including a handle 200 disposed at a proximal end 302 of a generally elongated shaft 300. An end effector 400 may be disposed at a distal end 304 of the shaft 300. The end effector 400 may include one or more jaws, such as a first jaw 502 and/or a second jaw 504, which may be disposed generally distally on a head 402. The handle 200 may include an actuator, such as a plunger 202, which may be operative to move the first jaw 502 and/or the second jaw 504 relative to the head 402, such as to close on (e.g., clamp) a target tissue 102.

As used herein with respect to FIGS. 6-11, “proximal” may refer generally to the direction towards the handle 200 end of the surgical clamp 100. As used herein with respect to FIGS. 6-11, “distal” may refer generally to the direction towards the end effector 400 end of the surgical clamp 100.

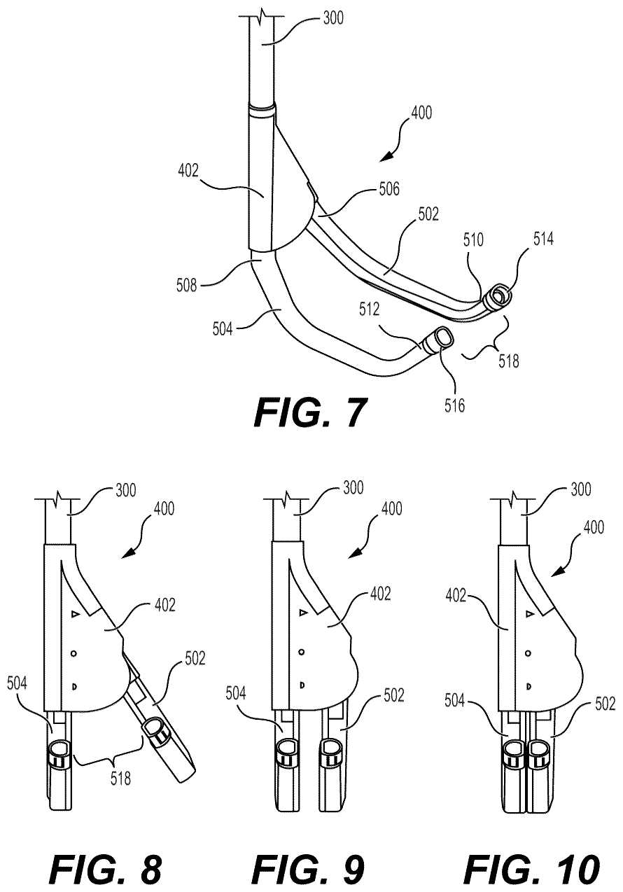

FIG. 7 is a detailed perspective view of an example end effector 400, according to at least some aspects of the present disclosure. Each of the first jaw 502 and the second jaw 504 may include a respective first end portion 506, 508 proximate the head 402 and a respective second end portion 510, 512 generally away from the head 402. Each of the second end portions 510, 512 may terminate at a respective tip 514, 516. In the open position of FIG. 7, the jaws 502, 504 may define an open mouth 518 between the spaced-apart tips 514, 516.

FIG. 8 is a side view of an example end effector 400 with the jaws 502, 504 in an open position, FIG. 9 is a side view of an example end effector 400 with the jaws 502, 504 in an intermediate position, FIG. 10 is a side view of an example end effector 400 with the jaws 502, 504 in a closed position, all in accordance with at least some aspects of the present disclosure.

Referring to FIGS. 6-8, in the open position, the first jaw 502 and second jaw 504 may be separated and may be substantially non-parallel. Referring to FIG. 9, in the intermediate position, the first jaw 502 and the second jaw 504 may be separated and may be substantially parallel. Referring to FIG. 10, in the closed position, the first jaw 502 and the second jaw 504 may be substantially adjacent and may be substantially parallel. As used herein with reference to the jaws 502, 504 in the closed position, “substantially adjacent” may include a small gap between the jaws 502, 504, such as due to the thickness of the target tissue 102 (FIG. 6), which may be clamped between the jaws 502, 504.

Referring to FIGS. 8-10, movement of the first jaw 502 from the open position (FIG. 8) to the intermediate position (FIG. 9) may include a substantial angular change (e.g., pivoting or rotating) with respect to the head 402, such as about an axis of rotation that is generally perpendicular to the shaft 300. Movement of the first jaw 502 from the intermediate position (FIG. 9) to the closed position (FIG. 10) may include a substantial translation with respect to the head 402, such as while the first jaw 502 and the second jaw 504 remain substantially parallel.

In some example embodiments, the first jaw 502 may be movable with respect to the head 402 while the second jaw 504 may be fixed (e.g., rigid) with respect to the head 402. In some circumstances, having one rigid jaw 504 may be advantageous because it may provide the surgeon with a fixed, known point of reference when positioning the clamp 100 (see FIG. 6). In other example embodiments, both the first jaw 502 and the second jaw 504 may be movable with respect to the head 402. For purposes of clarity and brevity, the description herein focuses on the movement of the first jaw 502 and the associated components facilitating such movement. A person of skill in the art will understand, however, that substantially similar components may be used to facilitate movement of the second jaw 504, thereby providing an alternative example embodiment in which both the first jaw 502 and the second jaw 504 may be movable with respect to the head 402 between the open position, the intermediate position, and the closed position, and such an embodiment is within the scope of this disclosure.

In some example embodiments, the shaft 300 may be substantially rigid. In other example embodiments, at least a portion of the shaft 300 may be bendable or malleable (e.g., plastically deformable), which may allow a user to configure the shaft 300 to accommodate a patient's specific anatomy. In some example embodiments, the shaft may be substantially straight (e.g., linear). In other example embodiments, the shaft 300 may include at least one curved portion. For example, the shaft 300 may be generally C-shaped (e.g., one curve) or S-shaped (e.g., two curves in opposite directions).

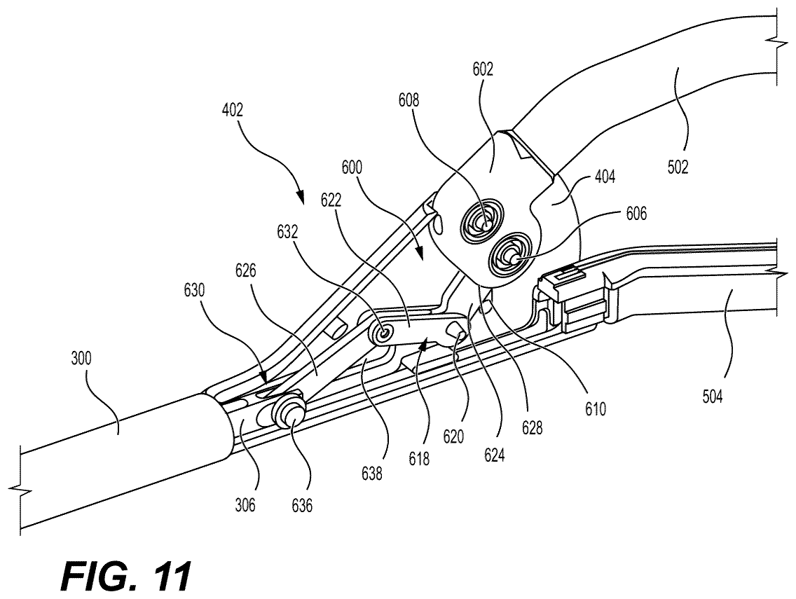

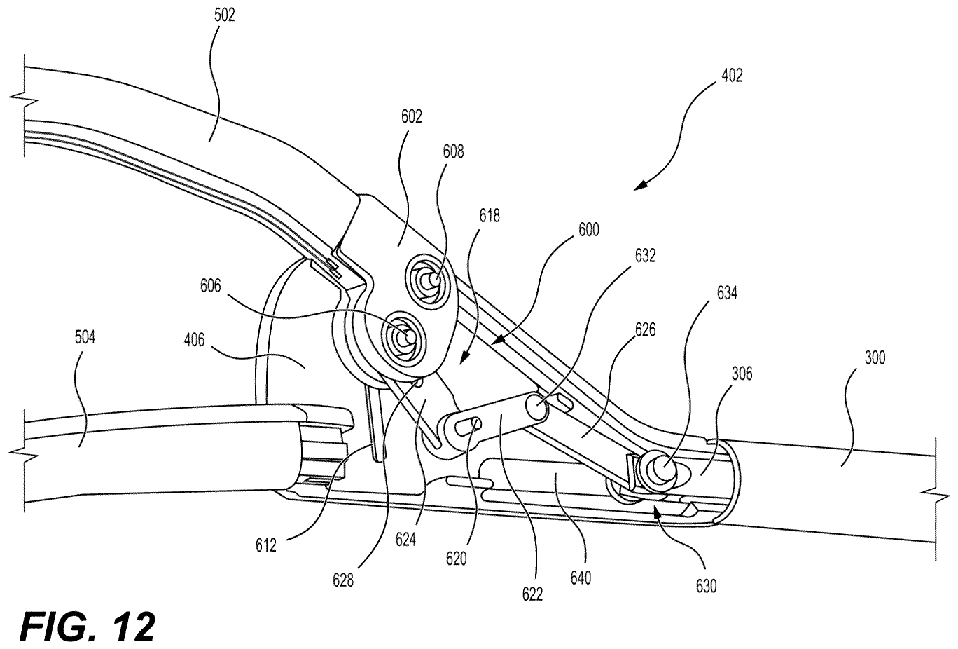

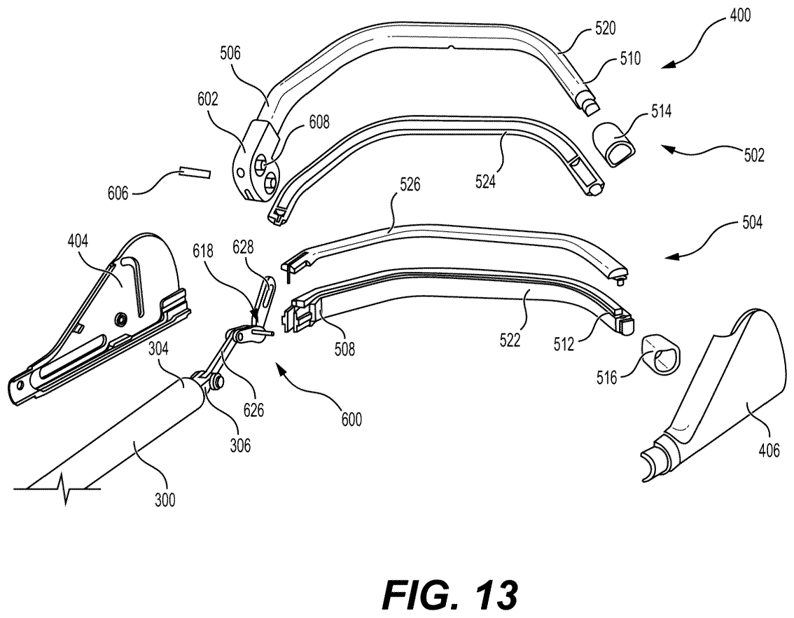

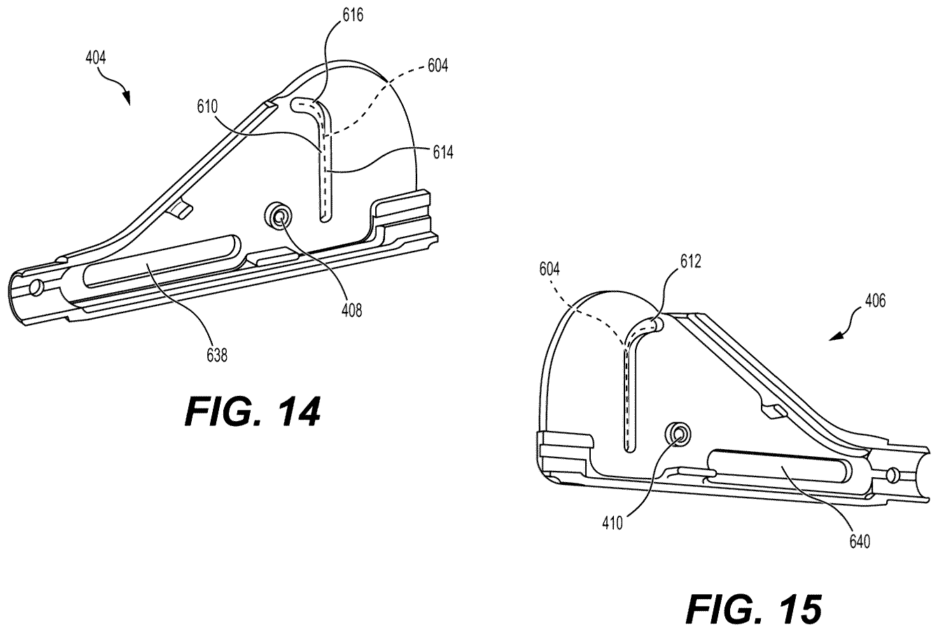

FIGS. 11 and 12 are perspective cutaway views of an example articulating mechanism 600 (FIGS. 11 and 12 are different side perspective views of the same example device), FIG. 13 is an exploded perspective view of an example end effector 400 including an example articulating mechanism 600, FIGS. 14 and 15 are detailed internal perspective views of example head shell portions 404, 406, all according to at least some aspects of the present disclosure. Generally, the articulating mechanism 600 may be operable to move the first jaw 502 between the open position, the intermediate position, and the closed position upon operation of the actuator 202 (FIG. 6) by the user. In embodiments including a movable second jaw 504, a similar articulating mechanism may be utilized in connection with the second jaw 504.

Referring to FIGS. 11-15, an example articulating mechanism 600 may include a first jaw mount 602, which may be coupled (e.g., rigidly affixed) to the first jaw 502. In some example embodiments, the first jaw mount 602 may be integrally formed with at least a portion of the first jaw 502, such as is shown in FIG. 13. In other embodiments, the first jaw 502 may be affixed to a separate component comprising the first jaw mount 602.

In some example embodiments, the first jaw mount 602 may be movable along a path 604 (FIGS. 14 and 15) with respect to the head 402, which may cause rotation and/or translation of the first jaw mount 602 and the first jaw 502 between the open position, the intermediate position, and/or the closed position. For example, the first jaw 502 may rotate about 45 degrees between the open position and the intermediate position and/or the first jaw may translate about 10 mm between the intermediate position and the closed position. For example, the first jaw mount 602 may include a first pin 606 and/or a second pin 608, which may be slidably and/or rotatably movable along the path 604, which may be at least partially defined one or more slots. For example, the path 604 may be at least partially defined by a slot 610 on an internal surface of the shell portion 404 of the head 402 and/or the path 604 may be at least partially defined by a slot 612 on an internal surface of the shell portion 406 of the head 402. Although the illustrated embodiment utilizes pins 606, 608 movable within slots 610, 612 to facilitate movement of the first jaw mount 602 along the path 604, it is within the scope of the disclosure to utilize other components and/or mechanisms, such as tracks, rollers, sliders, etc. to facilitate movement of the first jaw mount 602 along the path 604.

Referring to FIGS. 14 and 15, in some example embodiments, the path 604 (and/or the slots 610, 612) may comprise at least one generally straight portion 614 and/or at least one generally curved portion 616. In some embodiments including two pins 606, 608 moving along the path 604 defined by slots 610, 612, the generally curved portion 616 may be operable cause the first jaw mount 602 (and attached first jaw 502) to pivot or rotate substantially with respect to the head 402. The length, orientation, and/or curvature of the generally curved portion 616 may establish the extent of the angular rotation of the first jaw mount 602 and/or the amount of translation of the first jaw mount 602 with respect to the head 402. Similarly, in some embodiments including two pins 606, 608 moving along the path 604 defined by slots 610, 612, the generally straight portion 616 may be operable to cause the first jaw mount 602 (and attached first jaw 502) to translate with respect to the head 402 without substantially changing the angle of the first jaw mount 602 with respect to the head 402. The length and/or orientation of the generally straight portion 614 may establish the extent of the translation of the first jaw mount 602 with respect to the head 402. It is within the scope of this disclosure to utilize any combination of generally curved portions 616 and/or generally straight portions 614 to provide a desired path 604 to obtain a desired movement of the first jaw mount 602 (and attached first jaw 502). For example, an alternative path may include a continuous curve that varies in curvature over its length. Or, for example, an alternative path may include two generally straight portions 614 interposed by a curved portion 616.

Referring to FIGS. 11-15, in some example embodiments, the articulating mechanism 600 may include a crank 618 pivotably mounted with respect to the head 402. For example, the crank 618 may be pivotably disposed within the head 402, such as by a pivot pin 620 received in pivot holes 408, 410 of the shell portions 404, 406, respectively. The crank 618 may be operably coupled to the first jaw mount 602 to move the first jaw mount 602 along the path 604. For example, rotation of the crank 618 may cause the first jaw mount 602 to move along the path 604.

In some example embodiments, the crank 618 may include a first arm 622, which may be operably coupled to an actuator linkage 306, and/or a second arm 624, which may be operably coupled to the first jaw mount 602. The actuator 202 (FIG. 6) may be operably coupled to the actuator linkage 306, which may extend generally longitudinally through the shaft 300. Some example embodiments may include a connecting linkage 626 interposing the actuator linkage 306 and the crank 618. The articulating mechanism 600 may be configured so that movement of the actuator 202 causes rotation of the crank 618 (e.g., via the actuator linkage 306 and/or the connecting linkage 626). As the crank 618 rotates, the second arm 624 may move the first jaw mount 602 along the path 604 to move the first jaw 502 between the open position, the intermediate position, and/or the closed position.

Although the crank 618 of the illustrated embodiment comprises two, generally separately extending arms 622, 624, it is within the scope of the disclosure to utilize a crank with arms that are not substantially separately formed. For example, such a crank may be generally in the form of a circular sector of about 120 degrees in which the area between the arms is at least partially continuous. In some example embodiments, connecting the arms 622, 624 together at positions radially distant from the axis of rotation may increase the strength of the crank 618, thereby increasing the maximum allowable torque and/or forces for a given material and thickness. In some example embodiments, varying the effective lengths of the arms 622, 624 (e.g., the radial distances between the pivot pin 620 and the first pin 606 and/or the pivot pin 620 and the pivotable connection 632 (described below)) may facilitate varying the maximum allowable torque and/or force.

In some example embodiments, the distance between the pivot axis of the crank 618 (e.g., pivot pin 620) and the path 604 (along which the first pin 606 moves) may vary over the length of the path 604. Accordingly, the second arm 624 of the crank 618 may be slidably and/or pivotably coupled to the first jaw mount 602. For example, the second arm 624 of the crank 618 may include a crank slot 628, which may slidably and/or pivotably receive the first pin 606 of the first jaw mount 602 so that the first pin 606 moves along the crank slot 628 as the first jaw mount 602 moves along the path 604. In some example embodiments, the crank slot 628 may be substantially straight and/or may be oriented substantially radially with respect to the axis of rotation of the crank 618 (e.g., pivot pin 620).

In some example embodiments including a connecting linkage 626, a proximal end of the connecting linkage 626 may be coupled to a distal end of the actuator linkage 306 by a pivotable connection 630. A distal end of the connecting linkage 626 may be coupled to the first arm 622 of the crank by a pivotable connection 632. The pivotable connection 630 between the distal end of the actuator linkage 306 and the proximal end of the connecting linkage 626 may include one or more guides 634, 636, which may be slidable within respective guide slots 638, 640 on internal surfaces of the shell portions 404, 406 of the head 402. In some example embodiments, the guide slots 638, 640 may be generally linear and/or may be positioned substantially axially with respect to the shaft 300 so that the actuator linkage 306 moves generally proximally and distally in substantially a straight line (e.g., generally in-line with the actuator linkage 306).

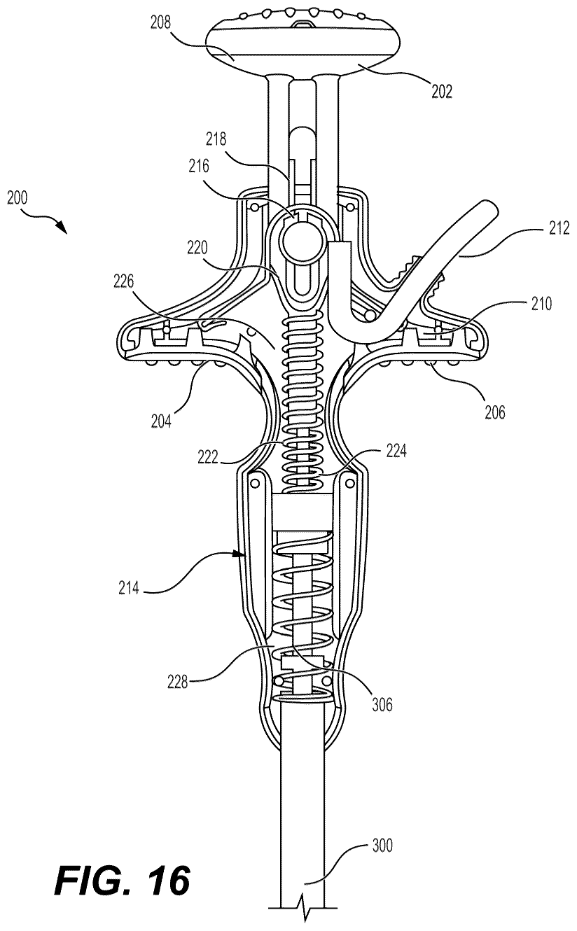

FIG. 16 is an internal side view of an example handle 200, according to at least some aspects of the present disclosure. Generally, the handle 200 may be constructed and/or may operate as described in U.S. Pat. No. 8,876,820, which is incorporated by reference. The handle 200 may include grips 204, 206, 208. The handle 200 may include a port 210 through which wires 212 or tubes may extend from the interior to the exterior of the handle 200. For instance, wires 212 for ablation electrodes or sensors on the jaws 502, 504 may be routed through the shaft 300, into the handle 200, and out through the port 210.

In some example embodiments, the handle 200 may house an actuator mechanism 214. In this example embodiment, the plunger 202 may be used to articulate one or more of the jaws 502, 504. The plunger 202 may be generally aligned with the shaft 300. With the plunger 202 in the fully retracted or proximal position, the first jaw 502 may be in the open position (see FIG. 8). When the plunger 202 is depressed in the distal direction, the first jaw 502 may move from the open position (FIG. 8) to the intermediate position (FIG. 9). Further depression of the plunger 202 may move the first jaw 502 from the intermediate position (FIG. 9) to the closed position (FIG. 10).

In some example embodiments, the actuator mechanism 214 may include a locking mechanism. For example, the plunger 202 may include a generally longitudinal slot 216 with a widened proximal opening 218. When the jaws 502, 504 are in the closed position, the opening 218 may align with a lock button 220, which may be spring-biased to drive the lock button 220 into the opening 218, thereby preventing the plunger 202 from moving proximally and maintaining the jaws 502, 504 in the closed position. Depressing the lock button 220 may disengage the lock button 220 from the opening 218, thereby releasing the plunger 202 and allowing it to move proximally to open the jaws 502, 504.

In some example embodiments, the actuator mechanism 214 may be configured to control and/or limit the amount of force that may be applied by the jaws 502, 504 when the plunger 202 is depressed. For example, the actuator mechanism 214 may include a relief rod 222 and a force limiting spring 224. The relief rod 222 may be slidable with respect to the actuator linkage 306, while the force limiting spring 224 may be arranged to apply a distal force to the actuator linkage 306. As the plunger 202 is depressed, the force limiting spring 224 may compressed between a step 226 on the plunger 202 and the actuator linkage 306. Accordingly, depressing the actuator 202 imparts a load on the force limiting spring 224 that is transferred to the actuator linkage 306, which moves the actuator linkage 306 distally. If the jaw clamping load exceeds the desired maximum while the plunger 202 continues to be depressed, the force limiting spring 224 is further compressed and the relief rod 222 moves distally without moving the actuator linkage 306. Thus, the force limiting spring 224 substantially limits the maximum jaw clamping load. One with ordinary skill in the art will recognize that the tissue clamping pressure may be a function of the jaw clamping force and the tissue area being clamped. The actuator mechanism 214 may include a return spring 228 that may be operative to move the actuator linkage 306 proximally upon releasing the actuator 202.

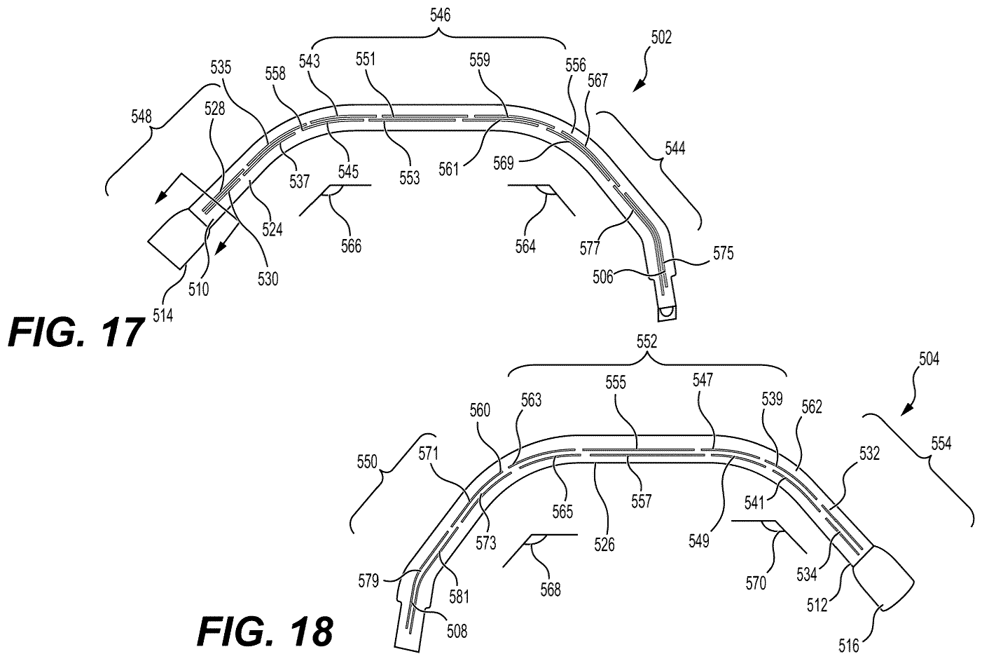

FIG. 17 is a detailed view of the inwardly facing (e.g., tissue-clamping) surface of an example first jaw 502, FIG. 18 is a detailed view of the inwardly facing (e.g., tissue-clamping) surface of an example second jaw 504, and FIG. 20 is a cross-sectional view of an example first jaw 502, all according to at least some aspects of the present disclosure. Although FIG. 20 illustrates certain components and dimensions associated with the first jaw 502, the second jaw 504 may include similar components with similar dimensions unless otherwise specifically indicated.

Referring to FIGS. 7, 13, 17, 18, and 20, in some example embodiments, the first jaw 502 may comprise a substantially rigid jaw beam 520 extending generally from the proximal end portion 506 (e.g., proximate the first jaw mount 602) to the distal end portion 510 (e.g., proximate the tip 514). Similarly, the second jaw 504 may comprise a substantially rigid jaw beam 522 extending generally from the proximal end portion 508 to the distal end portion 512 (e.g., proximate the tip 516). The jaw beams 520, 522 may be constructed from stainless steel, for example, which may provide the desired bending strength as well as acting as a heat sink during some procedures involving ablation. Other biocompatible materials providing suitable mechanical and thermal characteristics, such as other metals (e.g., aluminum), may be used for alternative jaw beams.

In some example embodiments, an insulator 524, 526 may be disposed on each respective jaw, such as on the inwardly facing surface of each respective jaw beam 520, 522. The insulators 524, 526 may be constructed of an electrically non-conductive material, such as molded plastic. Other biocompatible materials providing suitable insulative and thermal characteristics may be used for alternative insulators.

In some example embodiments, such as those configured for radio frequency (RF) ablation, the jaws 502, 504 may include two or more electrode pairs, which may be disposed on (e.g., mounted at least partially within) the insulators 524, 526. For example, electrode pairs may be bonded to or overmolded in the insulators 524, 526. The first jaw 502 may include two or more elongated, spaced apart electrode pairs, with electrodes 528, 530 comprising a first pair. Similarly, the second jaw 504 may include two or more elongated, spaced apart electrode pairs, with electrodes 532, 534 corresponding to the first pair of electrodes 528, 530 on the first jaw. In exemplary form, as depicted in FIGS. 17-19, the first and second jaws 502, 504 may include more than two segmented pairs of electrodes. By way of further example, each of the first and second jaws 502, 504 may include seven pairs of segmented electrodes. Namely, the first jaw 502 may include fourteen electrodes 528, 530, 535, 537, 543, 545, 551, 553, 559, 561, 567, 569, 575, 577, while the second jaw 504 may also include fourteen complementary electrodes 532, 534, 539, 541, 547, 549, 555, 557, 563, 565, 571, 573, 579, 581 (collectively, “segmented electrode pairs”). In exemplary form, the segmented electrode pairs may be configured to have a length, width, and shape that is aligned with its counterpart electrode so that the electrodes of the first jaw 502 each overlap a corresponding electrode of the second jaw 504. In exemplary form, the segmented electrode pairs may be configured to conduct bipolar, radio-frequency ablation of target tissue 102 (see FIG. 6) clamped between the jaws 502, 504.

As shown in FIG. 20, each electrode of the segmented electrode pairs may have a width 536, which may be the width of the tissue-facing surface in a direction generally perpendicular to the local, elongated direction of the electrode. Moreover, each electrode may be spaced apart by an electrode spacing 538. Each electrode may extend beyond the surface of the insulators 524, 526 by a projection height 540. Each electrode may be spaced at an insulation depth 542 from its respective jaw beam 520, 522. Each electrode may have an electrode height 572.

In some example embodiments, the electrode width 536 of the segmented electrode pairs may be about 0.1 mm to about 2.0 mm. In some example embodiments, the electrode width 536 may be about 0.2 to about 0.4 mm. In some example embodiments, the electrode width 536 may be about 0.30 mm. In some example embodiments, each electrode may have substantially the same width 536, which may be substantially constant over the length each electrode. In other embodiments, the width 536 may vary over the length of an electrode and/or may differ from electrode to electrode.

In some example embodiments, the widthwise electrode spacing 538 of the segmented electrode pairs may be about 0.1 mm to about 3.0 mm. In some example embodiments, the widthwise electrode spacing 538 may be about 0.3 mm to about 0.6 mm. In some example embodiments, the widthwise electrode spacing 538 may be about 0.43 mm. In some example embodiments, the widthwise electrode spacing 538 may be substantially constant over the length of the plurality of electrodes so that the electrodes are substantially parallel in any local region. In other embodiments, the widthwise electrode spacing 538 may vary over the length of the plurality of electrodes so that the electrodes may be closer together in some regions and/or farther apart in other regions. Generally, reducing the electrode spacing 538 may result in narrower lesions and/or faster ablation.

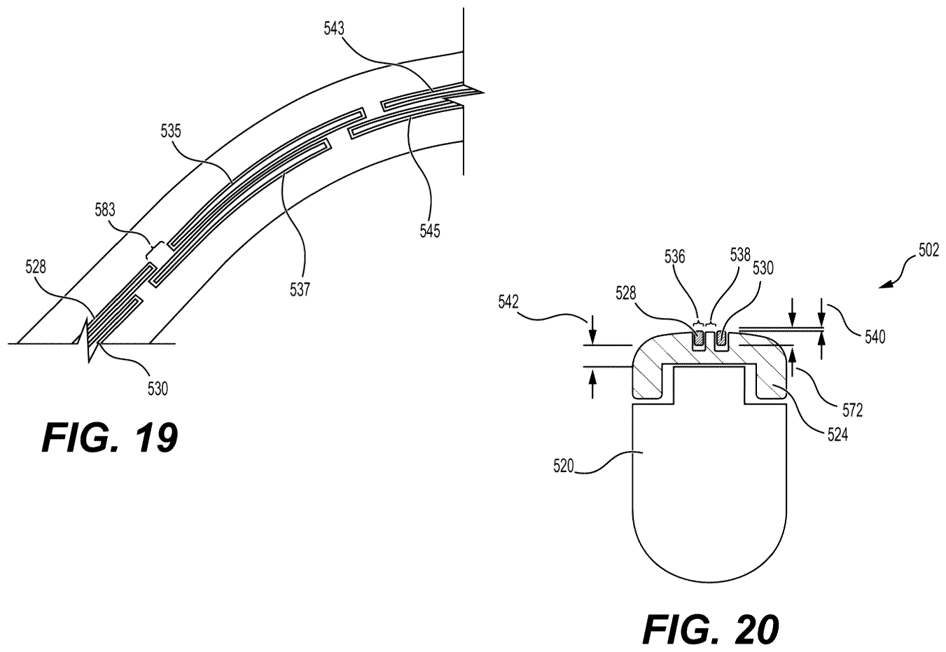

In some example embodiments, the longitudinal electrode spacing 583 (see FIG. 19) of the segmented electrode pairs may be about 0.1 mm to about 3.0 mm. In some example embodiments, the longitudinal electrode spacing 583 may be about 0.3 mm to about 0.6 mm. In some example embodiments, the longitudinal electrode spacing 583 may be about 0.43 mm. In some example embodiments, the longitudinal electrode spacing 583 may be substantially constant over the total length of the plurality of electrodes. In other embodiments, the longitudinal electrode spacing 583 may vary over the total length of the plurality of electrodes so that some electrodes may be closer together in some regions and/or farther apart in other regions.

In some example embodiments, the projection height 540 (see FIG. 20) may be about 0.0 mm (e.g., flush) to about 0.5 mm. In some example embodiments, the projection height 540 may be about 0.1 mm to about 0.2 mm. In some example embodiments, the projection height 540 may be about 0.15 mm. In some example embodiments, the projection height 540 may be substantially constant over the length of a particular electrode and/or may be substantially the same for two or more electrodes. In other embodiments, the projection height 540 may vary over the length of an electrode and/or from electrode to electrode.

In some example embodiments, the insulation depth 542 may be about 0.1 mm to about 5.0 mm. In some example embodiments, the insulation depth 542 may be about 0.8 mm to about 1.6 mm. In some example embodiments, the insulation depth 542 may be about 1.25 mm. In some example embodiments, the insulation depth 542 may be substantially constant over the length of a particular electrode and/or may be substantially the same for two or more electrodes. In other embodiments, the insulation depth 542 may vary over the length of an electrode and/or from electrode to electrode. Generally, increasing the insulation depth 542 may result in narrower lesions, faster ablation, and/or lower energy per unit volume.

In some example embodiments, the electrode height 572 may be about 0.25 mm to about 3.0 mm. In some example embodiments, the electrode height 572 may be about 0.3 mm to about 0.7 mm. In the example embodiments, the electrode height 572 may be about 0.5 mm. In some example embodiments, the electrode height 572 may be substantially constant over the length of a particular electrode and/or may be substantially the same for two or more electrodes. In other embodiments, the electrode height 572 may vary over the length of an electrode and/or from electrode to electrode.

In some example embodiments, the electrodes of the segmented electrode pairs may extend substantially the entire length of the jaws 502, 504 between the head 402 and the tips 514, 516. By way of example, the electrodes of the segmented electrode pairs on each jaw 502, 204 may have a total length of about 105 mm. In an alternative exemplary embodiment with shorter jaws, the electrodes of the segmented electrode pairs may have a total length of about 86 mm.

In some example embodiments according to at least some aspects of the present disclosure, the jaws 20, 22, 502, 504 of the clamp 12, 100 may be configured to facilitate positioning adjacent to and engagement of a particular target tissue 102 in a desired manner. For example, the shape of the jaws 20, 22, 502, 504 may be selected based on the target tissue 102 and/or the location of the target tissue 102 in relation other anatomical structures. As described in more detail hereafter, the foregoing clamps 12, 100 may be used to create lesions around the pulmonary veins, which are located generally on the posterior portion of the heart. When the heart is accessed via a median sternotomy, creating lesions around the pulmonary veins may require positioning the clamp 12, 100 at least partially around the posterior aspect of the heart, while engaging the left atrium and avoiding nearby structures that will not be ablated.

In some example embodiments, the jaws 20, 22, 502, 504 may be linear or substantially linear, or formed of one or more substantially straight (e.g., generally linear) portions 544, 546, 548, 550, 552, 554 (see FIGS. 17, 18), which may be interposed by one or more generally curved or bent portions 556, 558, 560, 562. For example, in the first jaw 502, a first substantially straight portion 544 may extend from the first end portion 506 to a first curved portion 556. A second substantially straight portion 546 may extend from the first curved portion 556 to a second curved portion 558. A third substantially straight portion 548 may extend from the second curved portion 558 to the second end portion 510. In some example embodiments, each of the substantially straight portions 544, 546, 548 may be obliquely oriented (e.g., non-parallel and non-perpendicular) with respect to each of the other substantially straight portions 544, 546, 548. For example, an angle 564 between the first substantially straight portion 544 and the second substantially straight portion 546 may be about 110 degrees to about 150 degrees and/or an angle 566 between the second substantially straight portion 546 and the third substantially straight portion 548 may be about 110 degrees to about 150 degrees. In the example embodiment shown in FIG. 17, portion 544 may have a length of about 2.9 cm, portion 546 may have a length of about 5.0 cm, portion 548 may have a length of about 2.8 cm, angle 564 may be about 128 degrees and/or angle 566 may be about 133 degrees.

Similarly, in the second jaw 504, a first substantially straight portion 550 may extend from the first end portion 508 to a first curved portion 560. A second substantially straight portion 552 may extend from the first curved portion 560 to a second curved portion 562. A third substantially straight portion 554 may extend from the second curved portion 562 to the second end portion 512. In some example embodiments, each of the substantially straight portions 550, 552, 554 may be obliquely oriented (e.g., non-parallel and non-perpendicular) with respect to each of the other substantially straight portions 550, 552, 554. For example, an angle 568 between the first substantially straight portion 550 and the second substantially straight portion 552 may be about 110 degrees to about 150 degrees and/or an angle 570 between the second substantially straight portion 552 and the third substantially straight portion 554 may be about 110 degrees to about 150 degrees. In the example embodiment shown in FIG. 18, portion 550 may have a length of about 2.9 cm, portion 552 may have a length of about 5.0 cm, portion 554 may have a length of about 2.8 cm, angle 568 may be about 128 degrees and/or angle 570 may be about 133 degrees.

In an alternative exemplary embodiment with shorter jaws, portion 544 may have a length of less than about 2.9 cm, portion 546 may have a length of less than about 5.0 cm, portion 548 may have a length of less than about 2.8 cm, angle 564 may be about 128 degrees and/or angle 566 may be about 133 degrees. Similarly, portion 550 may have a length of less than about 2.9 cm, portion 552 may have a length of less than about 5.0 cm, portion 554 may have a length of less than about 2.8 cm, angle 568 may be about 128 degrees and/or angle 570 may be about 133 degrees.

In some exemplary embodiments, the first jaw 20, 502 and the second jaw 22, 504 may be generally shaped as mirror images of one another, which may facilitate clamping the target tissue 102 between the jaws 20, 22, 502, 504 over any portion of their lengths. In other embodiments, the first jaw 20, 502 and the second jaw 22, 504 may have different dimensions.

Referring to FIGS. 17 and 18, in some exemplary embodiments, the respective substantially straight portions 544, 546, 548 associated with the first jaw 502 may be substantially coplanar (e.g., they may lie in substantially the same plane). Similarly, the respective substantially straight portions 550, 552, 554 associated with the second jaw 504 may be substantially coplanar. In other example embodiments, at least one of the substantially straight portions 544, 546, 548, 550, 552, 554 associated with a particular jaw 502, 504 may be substantially non-coplanar with respect to another substantially straight portion 544, 546, 548, 550, 552, 554 associated with that particular jaw 502, 504.

In some example embodiments, the first and third substantially straight portions 544, 548, 550, 554 of each jaw 502, 504 may facilitate positioning of the clamp 100 at a desired position on a patient's anatomy. For example, the first and third substantially straight portions 544, 548, 550, 554 of each jaw 502, 504 may facilitate positioning of the clamp 100 on the posterior side of the heart (e.g., on the left atrium) because they may point somewhat anteriorly. As compared to fully curved jaws, the more aggressive anterior orientation of the first and third substantially straight portions 544, 548, 550, 554 may improve positioning of the clamp 100 around the heart from an anterior surgical access location (e.g., median sternotomy).

In some example embodiments, the second substantially straight portions 546, 552 of each jaw 502, 504 may facilitate the desired engagement of the clamp 100 with a target tissue. For example, the second substantially straight portions 546, 552 of each jaw 502, 504 may facilitate the engagement of the clamp 100 with the left atrium (e.g., generally around the pulmonary veins). As compared to some fully curved jaws, the second substantially straight portions 546, 552 may engage the left atrium generally in a straight line between the right pulmonary veins and the left pulmonary veins, which may position the clamping (and ablation) location on the left atrium generally anteriorly and/or may facilitate forming an effective, transmural lesion. In addition, the second substantially straight portions 546, 552 may be less likely to slip off of the posterior aspect of the left atrium as compared to some fully curved jaws.

In some exemplary embodiments, the jaws 502, 504 may disposed generally distally on the head 402 and/or may be oriented generally laterally from the head 402 (e.g., generally from the first end portions 506, 508 to the second end portions 510, 512). For example, the second substantially straight portions 546, 552 of each jaw 502, 504 may be oriented approximately perpendicularly to the shaft 300. In the exemplary embodiments, in the closed position, the second substantially straight portions 546, 552 may be oriented at an angle of about 98 degrees with respect to the shaft 300. In an alternative exemplary embodiment including shorter jaws, in the closed position, the second substantially straight portions 546, 552 may be oriented at an angle of about 98 degrees with respect to the shaft 300. In some other embodiments, in the closed position, the second substantially straight portions 546, 552 may be oriented at an angle of about 45 degrees to about 135 degrees with respect to the shaft 300. In other exemplary embodiments, the jaws 502, 504 may be oriented at other angles with respect to the shaft 300, such as generally in line with the shaft 300 (e.g., extending generally directly distally).

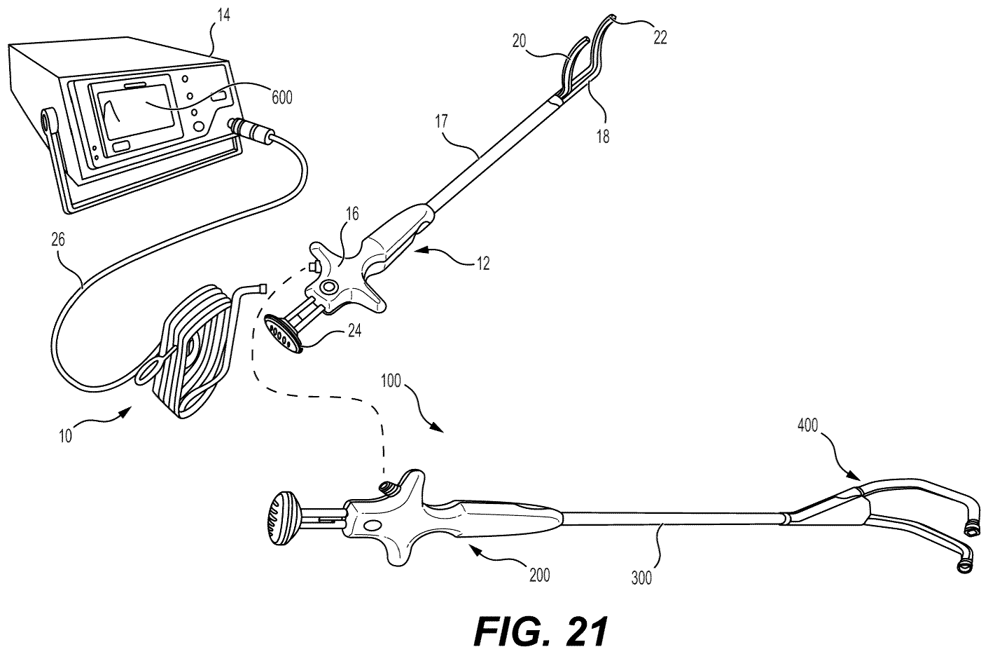

Turning to FIG. 21, utilization of the electrosurgical system 10 with either or both of the clamps 12, 100 may be effectuated to form one or more tissue lesions. In exemplary form, the each of the electrode pairs (21, 37) (31, 39) (28, 36) (30, 38) for the first exemplary clamp 12 and each of the electrode pairs (528, 532), (530, 534), (535, 539), (537, 541), (543, 547), (545, 549), (551, 555), (553, 557), (559, 563), (561, 565), (567, 571), (569, 573), (575, 579), (577, 581) for the second exemplary clamp 100 may be individually controlled or may be controlled as a group of two or more pairs of segmented electrodes using the control system/RF generator 14. Moreover, it is envisioned that the clamps 12, 100 are utilized to sandwich tissue 102 (see FIG. 6) to be ablated between respective clamp jaws including complementary electrodes of the segmented electrode pairs.

By way of example, in furtherance of carrying out an ablation, RF energy may be delivered from control system/RF generator 14 individually to each of a plurality of segmented electrode pairs or may be delivered and divided across two or more segmented electrode pairs. Unless otherwise noted, it is to be understood that if RF energy is delivered to a segmented electrode pair, this RF energy is not diminished, degraded, or otherwise eroded regardless of the number of segmented electrode pairs concurrently active.

In exemplary form, prior to carrying out an active tissue ablation, the control system/RF generator 14 may utilize one or more of the segmented electrode pairs, when tissue is clamped therebetween, to discern what the impedance of the clamped tissue is at predetermined intervals. By way of example, these predetermined intervals may correspond to the intervals of the locations of each of the segmented electrode pairs. Determining the impedance of clamped tissue may allow the control system/RF generator 14 to determine an RF energy for each segmented electrode pair that may be location specific. Put another way, by determining the tissue impedance at each of the segmented electrode pairs, the control system/RF generator 14 is able determine how much RF energy is delivered to each segmented electrode pair and whether two or more of the segmented electrode pairs will be effectively operated in parallel (dividing a singular RF energy across two or more segmented electrode pairs).

By way of example, the control system/RF generator 14 may initially direct alternating current through a first of the electrodes of each of the segmented electrode pairs when tissue 102 is clamped by the clamp 12, 100. This alternating current flows into the tissue 102 and the resulting voltage of this alternating current is measured by the second of the electrodes of each of the segmented electrode pairs. The resulting voltage drop across the tissue 102 is measured and the control system/RF generator 14 calculates the impedance using Ohm's law by combining resistance and reactance. Based upon programming within the control system/RF generator 14, a predetermined impedance threshold determines whether the segmented electrodes will be operated individually or in parallel (RF energy divided across two or more segmented electrodes). By way of example, if the determined impedance across a segmented electrode pair is less than the predetermined impedance threshold (such as, without limitation, 450 ohms), then that segmented electrode pair may be operated individually, meaning that RF power supplied to that segmented electrode pair will not be distributed to another segmented electrode pair. While a first segmented electrode pair may be driven at the same RF power rate as a second segmented electrode pair, the total RF power delivered to the first segmented electrode pair is not dependent upon another segmented electrode pair. For those segmented electrode pairs having an impedance below the predetermined impedance threshold, each segmented electrode pair may be driven individually and continuously by the control system/RF generator 14 until the measured impedance during the ablation across the electrodes of that segmented electrode pair meets or exceeds an ablation impedance threshold. Upon reaching or exceeding the ablation impedance threshold across a segmented electrode pair driven individually, the control system/RF generator 14 may continue RF power to that segmented electrode pair.

Alternatively, when the control system/RF generator 14 determines the impedance of tissue 102 across a segmented electrode pair meets or exceeds a predetermined impedance threshold, then the control system/RF generator 14 may distribute RF power concurrently across two or more segmented electrode pairs and operate these segmented electrodes as a single unit. By way of further explanation, the control system/RF generator 14 may group the segmented electrode pair (with an impedance meeting or exceeding the predetermined impedance threshold) with its closest adjacent segmented electrode pair to determine a new impedance of the tissue interposing these two segmented electrode pairs and determine whether this impedance exceeds the predetermined impedance threshold. If the new impedance of the grouped segmented electrode pairs does not exceed the predetermined impedance threshold, then the control system/RF generator 14 may operate the two segmented electrode pairs in parallel by distributing a single RF power supply across both segmented electrode pairs. Alternatively, if the new impedance determination across the two segmented electrode pairs exceeds the predetermined impedance threshold, then the control system/RF generator 14 may group these two segmented electrode pairs with the next nearest segmented electrode pair (to provide a group of three segmented electrode pairs) and thereafter determine the impedance of tissue between these three segmented electrode pairs to determine whether the new impedances exceeds the predetermined impedance threshold. If the new impedance determination for these three segmented electrode pairs operated as a group does not exceed the predetermined impedance threshold, then the control system/RF generator 14 may operate these three segmented electrode pairs as a group in parallel by distributing a single RF power supply across these three segmented electrode pairs. Alternatively, if the new impedance determination across these three segmented electrode pairs exceeds the predetermined impedance threshold, then the control system/RF generator 14 may include the next nearest segmented electrode pair as part of the group (now a group of four segmented electrode pairs) and this process repeats until the new impedance calculated from the tissue interposing the segmented electrode pairs grouped is below the predetermined impedance threshold (at which point the segmented electrode pairs where the new impedance is below the threshold will be operated together, as a single unit/group) or there are no more pairs of segmented electrodes. If there are no more segmented electrode pairs available because the threshold impedance is still exceeded for that group of segmented electrode pairs, the control system/RF generator 14 may revert to operating each of the segmented electrode pairs of that group individually. After determining whether each segmented electrode pair will be operated independently or operated in conjunction with one or more segmented electrode pairs, the control system/RF generator 14 may initialize the ablation sequence and power the segmented electrode pairs. The powering of all or fewer than all of the segmented electrode pairs may be accomplished simultaneously. In this fashion, the control system/RF generator 14 actively monitors the segmented electrode pairs to which RF power is supplied and continues the ablation across the segmented electrode pairs until reaching a predetermined ablation completion metric.

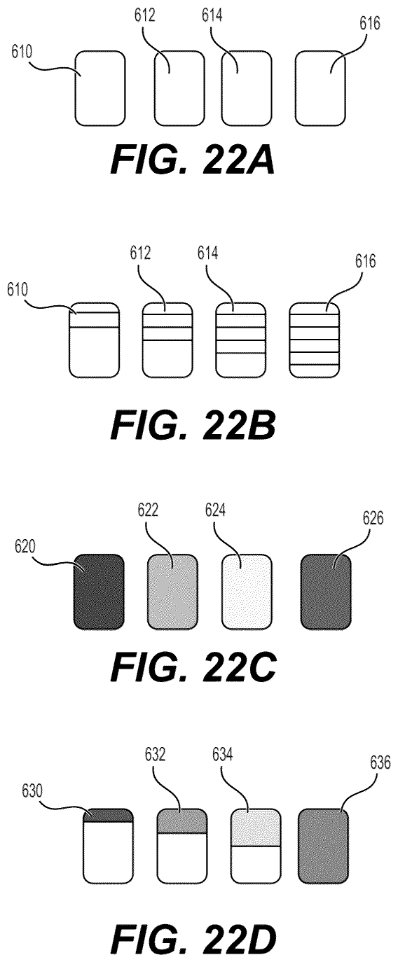

Referring to FIGS. 21, 22A, and 22B, it is within the scope of the disclosure that the control system/RF generator 14 includes a visual display 600 for displaying progress of the issue ablation when using either or both of the foregoing exemplary clamps 12, 100. By way of example, the system may include a visual display 600 including colors, indicia, numbers, and various other forms of visual information indicative of the progress on achieving lesion formation between corresponding electrode pairs. In a context where the clamp 12, 100 includes five pairs of segmented electrodes that are configured to be energized to effectuate ablation of tissue between the pairs of electrodes, the visual display 600 may include five rounded squares, with each rounded square corresponding to one of the electrode pairs. Though not required, the rounded squares may sequentially represent each of the five segmented electrode pairs in its sequence on the clamp. For example, the electrode pair nearest the distal tip of the clamp 12, 100 may be designated electrode pair #1 and the first rounded square on the far left of the display may correspond to electrode pair #1. Similarly, the electrode pair immediately proximal to electrode pair #1 on the clamp may be designated as electrode pair #2, and the second rounded square immediately to the right of first rounded square may correspond to electrode pair #2. This same correspondence can generally continue so that the order of the electrode pairs on the clamp is maintained as part of the visual display. It should also be noted that any other geometric shape such as, without limitation, a circle, a square, a rectangle, and a triangle may be used in lieu of or in addition to a rounded rectangle to represent each electrode pair. Moreover, it is also within the scope of the disclosure to use any other image or indicia to provide visual feedback to a user of the control system/RF generator 14 as to the progress of the ablation procedure, where the image or indicia is associated with one or more of the segmented electrode pairs.

By way of example, FIGS. 22A and 22B include four rounded rectangles, with each of the rounded rectangles 610-616 representing each of four segmented electrode pairs. For purposes of explanation, the four segmented electrode pairs may correspond to electrode pairs (21, 37) (31, 39) (28, 36) (30, 38) for the first exemplary clamp 12 or electrode pairs (528, 532), (530, 534), (535, 539), (537, 541) for the second exemplary clamp 100. In one exemplary configuration, each rounded rectangle may be initially depicted as an having an internal void. During the course of powering the respective segmented electrode pairs, the interior of the rounded rectangles 610-616 may be updated, optionally in real-time, with indicia reflecting the relative status of the ablation.

One alternative of this updated interior is depicted in FIG. 22B, where one or more horizontal bars are visible showing progress of the ablation specific to each segmented electrode pair. By way of example, in a circumstance where the first rounded rectangle 610 corresponds to a first segmented electrode pair, and the ablation is between 40-59 percent complete, the first rounded rectangle may include a first horizontal bar and a second horizontal bar. As the ablation continues and passes beyond 59 percent completion, but less than 79 percent completion, a third horizontal bar may be added. The second rounded rectangle 612 shows the status of an ablation when the ablation is between 60-79 percent complete. It should be noted, however, that the second rounded rectangle 612 may be concurrently visually depicted along with the first, third, and fourth rounded rectangles 610, 614, 616, and that the visual indication of the second rounded rectangle is specific to a second segmented electrode pair. In a circumstance where the ablation is between 80-99 percent complete, a fourth horizontal line may be depicted within the rounded rectangle. By way of example, the third rounded rectangle represents an ablation that is between 80-99 percent complete with respect to a third segmented electrode pair. When the ablation reaches completion, a fifth horizontal line may be added to the interior of the rounded rectangle. The fourth rounded rectangle 616 depicts a configuration where the ablation of the fourth segmented electrode pair is complete and includes five horizontal lines. Though not required, completion of the ablation may be achieved when the ablation is transmural. While the foregoing description describes how additive horizontal lines occupying the interior of a respective the rounded rectangle 610-616 may be used to show the relative progress of an ablation occurring between a segmented electrode pair, it is also within the scope of the disclosure to use other visual indications to provide feedback to a user regarding the progress of an ablation occurring.

Turning to FIG. 22C, it is also within the scope of the disclosure to use color to provide a visual indication as to the progress of an ablation occurring between a respective pair of segmented electrodes. As is the case with respect to FIG. 22A, each of the four rounded rectangles 620-626 represents a separate one of four segmented electrode pairs. By way of example, in a circumstance where the first rounded rectangle 620 corresponds to a first segmented electrode pair, and the ablation is less than 25 percent complete, the first rounded rectangle may be filled with a red area. As the ablation continues and passes beyond 25 percent completion, but less than 50 percent completion, the red area may be supplanted by an orange area. The second rounded rectangle 622 shows the status of an ablation corresponding to a second segmented electrode pair when the ablation is between 25-49 percent complete. Again, it should be noted that the second rounded rectangle 622 may be concurrently visually depicted along with the first, third, and fourth rounded rectangles 620, 624, 626, and that the visual indication of the second rounded rectangle is specific to a second segmented electrode pair. In a circumstance where the ablation passes beyond 50 percent completion, but less than 75 percent completion, the orange area may be supplanted by a yellow area. The third rounded rectangle 624 shows the status of an ablation corresponding to a third segmented electrode pair when the ablation is between 50-74 percent complete. Again, it should be noted that the third rounded rectangle 624 may be concurrently visually depicted with the other rounded rectangles. Where the ablation passes beyond 75 percent completion, but less than 100 percent completion, the orange area may be supplanted by light green area (similar to the light green color shown in the fourth rounded rectangle 636 of FIG. 22D). As depicted by the fourth rounded rectangle 626, when the ablation reaches 100 percent completion, the light green area may be supplanted by a dark green area. Though not required, completion of the ablation may be achieved when the ablation is transmural.

Referring to FIG. 22D, in a circumstance where the first rounded rectangle 630 corresponds to a first segmented electrode pair, and the ablation is less than 25 percent complete, the first rounded rectangle may be partially filled with a red area occupying less than 25 percent of the interior of the first rounded rectangle. As the ablation continues and passes beyond 25 percent completion, but less than 50 percent completion, the red area may be supplanted by an orange area occupying less than 50 percent of the interior of the first rounded rectangle. The second rounded rectangle 632 may show the status of an ablation corresponding to a second segmented electrode pair when the ablation is between 25-49 percent complete. Again, it should be noted that the second rounded rectangle 632 may be concurrently visually depicted along with the first, third, and fourth rounded rectangles 630, 634, 636, and that the visual indication of the second rounded rectangle is specific to a second segmented electrode pair. In a circumstance where the ablation passes beyond 50 percent completion, but less than 75 percent completion, the orange area may be supplanted by a yellow area occupying less than 75 percent of the interior of the rounded rectangle. The third rounded rectangle 634 shows the status of an ablation corresponding to a third segmented electrode pair when the ablation is between 50-74 percent complete. Again, it should be noted that the third rounded rectangle 634 may be concurrently visually depicted with the other rounded rectangles. As depicted by the fourth rounded rectangle 636, when the ablation reaches 75 percent, but is less than 100 percent, the yellow area may be supplanted by a light green area occupying more than 75 percent of the interior of the fourth rounded rectangle. And when the ablation is complete, this light green area may be supplanted by a dark green area (similar to the dark green color shown in the fourth rounded rectangle 626 of FIG. 22C) that occupies the entire interior of the rounded rectangle, thereby evidencing to a user that the ablation is finished as to that segmented electrode pair. Though not required, completion of the ablation may be achieved when the ablation is transmural.

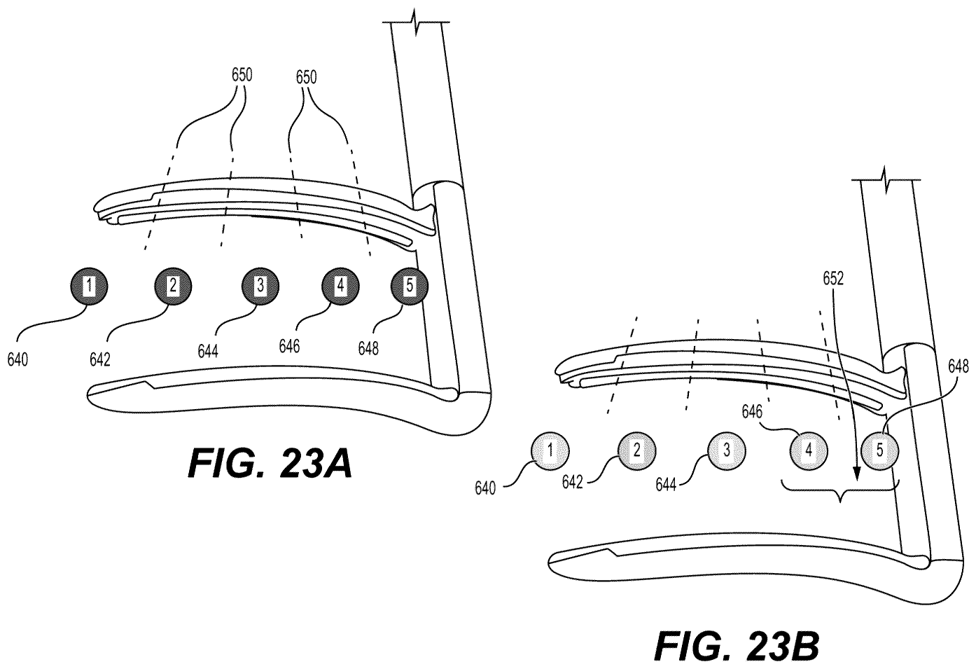

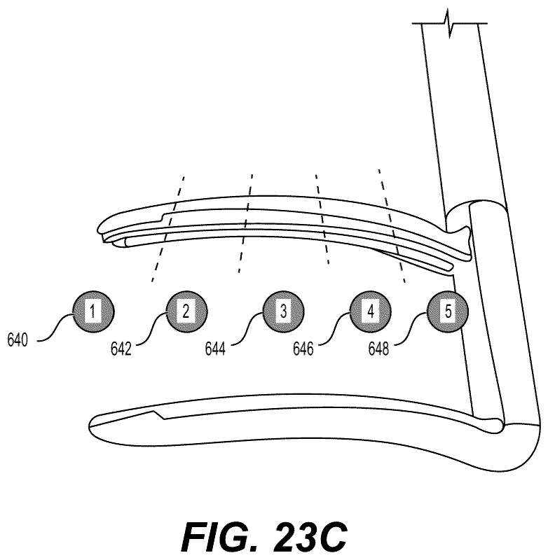

As depicted in FIGS. 23A-23C, it is it is also within the scope of the disclosure to use color and optionally to provide a visual representation of the clamp's working end (such as jaws) to visually represent the progress of a tissue ablation occurring between a respective pair of segmented electrodes or several pairs of segmented electrodes. In exemplary form, the depiction may include two or more segmented electrode pairs, such as five segmented electrode pairs. For each pair of segmented electrodes, a circle with a corresponding number is depicted. Here, there are five circles 640-648, with each circle corresponding one of five separate segmented electrode pairs distributed across two clamp jaws. In addition, there are a series of four dotted lines 650 showing the approximate boundaries between the respective segmented electrode pairs (essentially demarcating separate coverage areas of the five segmented electrode pairs). In exemplary form, the segmented electrode pairs may have terminal, longitudinal boundaries that fall short of one another, abut one another, and/or longitudinally overlap one another. Initially, all five circles are colored red, with the red color being indicative of an able ablation that has yet to start or one that has started and is less than a certain percentage complete (for example, 25 percent complete). As the ablation commences or continues, the color of the circles 640-648 may periodically change to reflect the progress of the ablation. By way of example, the segmented electrode pairs may measure changes in voltage indicative of changes in impedance of the tissue being ablated during the ablation, with the control system/RF generator 14 being operative to calculate the impedance changes in real time and use these impedance determinations to calculate a percent completion or other metric for measuring progress of the ablation sequence. Though not limiting, the color changes may go from red to orange, orange to yellow, yellow to light green, and light green to dark green, for example, to correspond to the start of the ablation, continuing with the ablation, and completion of the ablation. Though not required, completion of the ablation may be achieved when the ablation is transmural. In this fashion, the user of the ablation system can readily determine the stage of the ablation at each segmented electrode pair, as well as the relative location of each segmented electrode pair. Not only is the progress and location visually represented, but so too are segmented electrode pairs that are being operated as a unit.

As referenced herein, in certain circumstances, two or more segmented electrode pairs may be operated together. What this means is that power supplied by the control system/RF generator 14 is evenly distributed across each of the segmented electrode pairs being operated together. FIG. 23B includes a bracket 652, which visually confirms to a user of the ablation system that the fourth and fifth pairs of segmented electrodes are being powered as one unit. As a result, the fourth and fifth circles 646, 648 will always be colored similarly so that as the ablation progresses, and the circles 646, 648 change in color, this color change will occur at the same time, indicative of these segmented electrode pairs being operated as one unit (as one segmented electrode pair). When all five of the segmented electrode pairs reach ablation completion, the circles 640-648 may all be colored dark green, as shown in FIG. 23C, to visually confirm to a user that the ablation is complete.

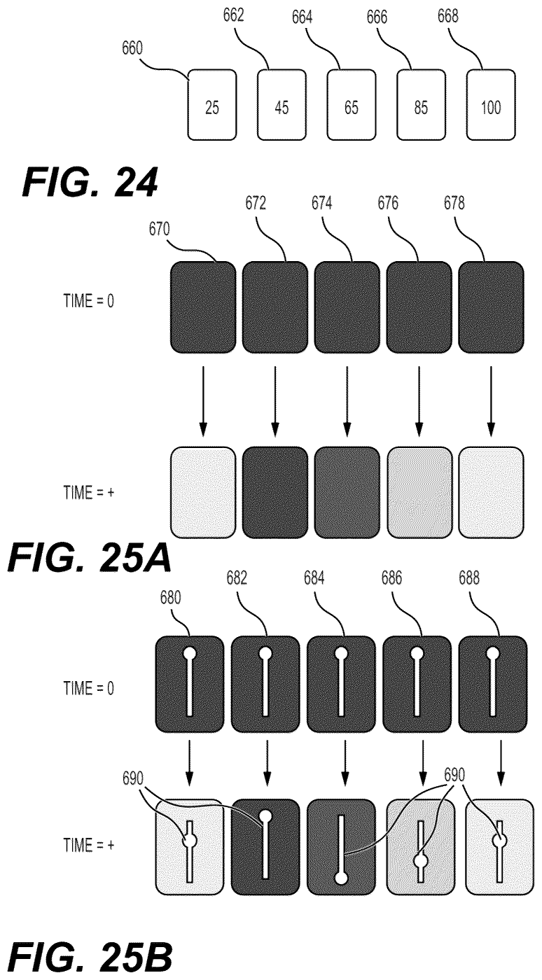

Referring to FIG. 24, it is also within the scope of the disclosure to provide a visual numerical representation of two or more segmented electrode pairs (such as jaws) to visually represent the progress of a tissue ablation occurring. In exemplary form, the depiction may include two or more segmented electrode pairs, such as five segmented electrode pairs. For each pair of segmented electrodes, a rounded rectangle with a corresponding number is depicted. Here, there are five rounded rectangles 660-668, with each rounded rectangle corresponding one of five separate segmented electrode pairs distributed across two clamp jaws, for example. As with other embodiments, the segmented electrode pairs may have terminal, longitudinal boundaries that fall short of one another, abut one another, and/or longitudinally overlap one another. Initially, all five rounded rectangles may start with a zero “0”, evidencing that the ablation has yet to start or has just started. As the ablation commences or continues, the number inside each of the circles 660-668 may periodically change to reflect the progress of the ablation. By way of example, the segmented electrode pairs may measure changes in voltage indicative of changes in impedance of the tissue being ablated during the ablation, with the control system/RF generator 14 being operative to calculate the impedance changes in real time and use these impedance determinations to calculate a percent completion or other metric for measuring progress of the ablation sequence. Though not limiting, the numerical changes may increase in hole number values from zero to one hundred, with changes being represented in any iteration desired (including percentage changes by a tenth of a percent, a percent, five percent, ten percent, etc.). Though not required, completion of the ablation may be achieved when the ablation is transmural. In this fashion, the user of the ablation system can readily determine the stage of the ablation at each segmented electrode pair, as well as the relative location of each segmented electrode pair presuming the five rounded rectangles are arranged from left to right as the segmented electrodes are generally arranged longitudinally along the clamp 12, 100. Though not represented in FIG. 24, a bracket may also be included to identify those segmented electrode pairs that are being operated as a unit.

Referencing FIG. 25A, a further alternate exemplary display may include a series of rounded rectangles 670-678, with each of the rounded rectangles corresponding to a separate segmented electrode pair (or two or more segmented electrode pairs being operated as a unit) of an ablation device, such as a clamp 12, 100. In this alternate exemplary display, each of the rounded rectangle is initially colored dark blue before and just prior to energizing any of the segmented electrode pairs. At a later time, after the ablation sequence is started, denoted by “Time=+”, each of the rounded rectangles 670-678 may change its colors to reflect the status of the ablation with respect to a separate segmented electrode pair or two or more segmented electrode pairs being operated as a unit. In exemplary form, as evidenced by the first and fifth rounded rectangles 670, 678, each rounded rectangle is colored a light blue evidencing the ablation has been initiated for those corresponding segmented electrode pairs, but has not reached a first predetermined stage, such as the ablation being 40 percent complete. While not reaching the first predetermined stage, the ablation segmented electrode pairs may continue to be powered. The second rounded rectangle 672, still having a dark blue color at Time=+ indicates the corresponding segmented electrode pair has not yet been powered and the ablation not yet initiated. But the third rounded rectangle 674, exhibiting a dark green color at Time=+ indicates the corresponding segmented electrode pair has been powered off because the ablation is completed for this segmented electrode pair. Though not required, completion of the ablation may be achieved when the ablation is transmural. Finally, the fourth rounded rectangle 676 exhibits a light green color at Time=+ indicating the corresponding segmented electrode pair continues to be powered after reaching or exceeding the first predetermined stage, but the ablation has not yet reached completion (as evidenced by a dark green color).

Referring to FIG. 25B, a further alternate exemplary display having all or some of the features described in FIG. 25A may also include a include a series of rounded rectangles 680-688, where each of the rounded rectangles (corresponding to a separate segmented electrode pair or two or more segmented electrode pairs being operated as a unit of an ablation device, such as a clamp 12, 100) may also include a vertical slider 690. By way of example, this vertical slider may include a white circle positioned along a white thin vertical rectangle. Depending upon the vertical position of the circle relative to the thin vertical rectangle, a user can immediately assess the progress of an ablation for the corresponding segmented electrode pair(s). For instance, when the white circle is near the top of the thin vertical rectangle, as shown by the first and fifth rounded rectangles 680, 688 at Time=+, the user is being told that the ablation is underway for those corresponding segmented electrode pairs, but that the ablation is not more than 50 percent complete for either segmented electrode pair. This vertical slider 690 provides general feedback to the user regarding progress of the ablation that is teamed with a color change of the rounded rectangles 680, 688, in this case from dark blue to light blue. Conversely, at Time=+ for the second rounded rectangle 682, the color remains dark blue and the white circle is positioned at the top of the thin vertical rectangle, which signals a user that the ablation has not yet commenced for this corresponding segmented electrode pair. With respect to the third rounded rectangle 684 at Time=+, the color of the rounded rectangle is dark green and the white circle of the vertical slider 690 is at the bottom of the thin vertical rectangle. This indicia of the third rounded rectangle 684 is telling a user that the ablation is complete and the corresponding segmented electrode pair is no longer powered on. Though not required, completion of the ablation may be achieved when the ablation is transmural. Finally, with respect to the fourth rounded rectangle 686 at Time=+, the color of the rounded rectangle is light green and the white circle of the vertical slider 690 is nearer the bottom of the thin vertical rectangle than the top of the thin vertical rectangle. This indicia of the fourth rounded rectangle 686 is telling a user that the ablation is approaching completion and may be more than 50 percent complete, as well as the fact that the corresponding segmented electrode pair is continuing to be powered. As the ablation progresses, the colors of the rounded rectangles 680-688 and disposition of the vertical slider 690 may be updated in real-time to reflect the progress of the ablation at the corresponding segmented electrode pairs.

While the foregoing graphical shapes, such as rounded rectangles and circles, have been described and depicted, it should be understood that any other shape may be incorporated, whether the shapes are uniform or not. For instance, the first electrode pair may be represented by a circle, the second electrode pair may be represented by a triangle, the third electrode pair may be represented by a trapezoid, the fourth electrode pair may be represented by an oval, and the fifth electrode pair may be represented by a rectangle, or various combinations thereof or substitutions of various shapes. Moreover, the graphical shapes may be tied to the number of operable electrode pairs of the medical device in question or may not be. For example, where two or more segmented electrode pairs are operated and powered as a single unit, the graphical shape for these segmented electrode pairs may differ from the graphical shape representing those segmented electrode pairs being operated and powered individually. For instance, a segmented electrode pair operated individually may be represented by a rounded rectangle, while a segmented electrode pair operated and distributively powered with other segmented electrode pairs may be represented by a triangle. Those skilled in the art will appreciate and understand the various options available in light of the foregoing disclosure.

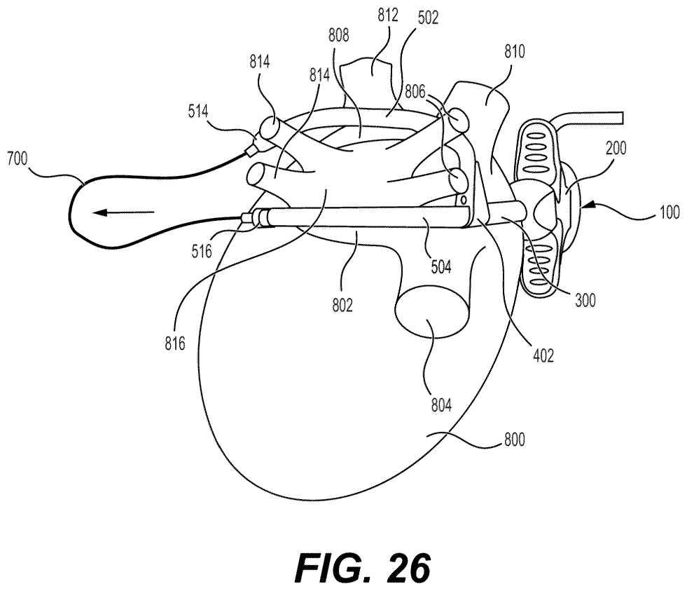

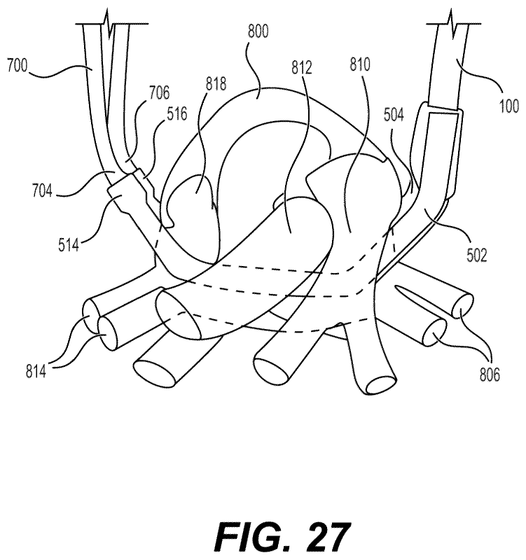

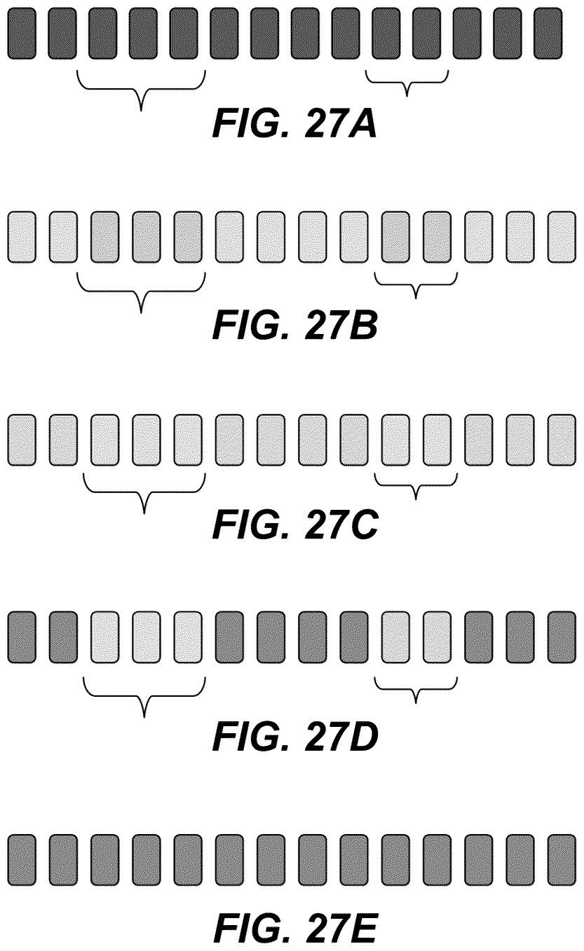

Referring to FIGS. 26 and 27, an example method of using two or more pairs of segmented electrodes will be discussed hereafter. Though not limiting, the exemplary method will describe use of the second exemplary clamp 100. But it should be understood that the first exemplary clamp 12 may be substituted for the second exemplary clamp in the following exemplary method discussion.

In exemplar form, one or more incisions may be made with respect to a patient to access the area in proximity to the heart 800. Thereafter, the ablation clamp 100 may be introduced with first and second jaws 502, 504 closed through an incision, such as through a trocar occupying part of the incision, and thereafter moving the clamp 100 from the anterior, right side of the patient's heart 800. The jaws 502, 504 of the clamp 100 may be at least partially opened so that at least a portion of jaws is interposed by the pulmonary veins 806, 814. A flexible guide 700 may include opposing end portions 704, 706 configured to releasably engage corresponding tips 514, 516 of the first and second jaw 502, 504 of the clamp 100. In this fashion, the end portions 704, 706 of the guide may be releasably attached to the corresponding tips 514, 516 of the first and second jaw 502, 504 so that a portion of the guide extends on top and bottom of the pulmonary veins 806, 814.

Referring to FIG. 26, the guide 700 may be pulled generally toward the patient's left, which may pull the jaws 502, 504 of the clamp 100 into a position near the pulmonary veins 806, 814 and/or the left atrium 816. The clamp handle 200 may be generally anteriorly positioned, while the shaft 300 may extend generally posteriorly along the right side of the heart 800. The clamp head 402 may be positioned on the right, posterior aspect of the heart 800, generally to the right of the right pulmonary veins 806. The tips 514, 516 of the jaws 502, 504 may be generally to the left of the left pulmonary veins 814. The first jaw 502 may extend generally from right to left, generally anterior and superior to the pulmonary veins 806, 814 and posterior to the superior vena cava 810 and aorta 812 (e.g., generally through the transverse sinus 808). The second jaw 504 may extend generally from right to left, generally anterior and inferior to the pulmonary veins 806, 814 and superior to the inferior vena cava 804 (e.g., generally through the oblique sinus 802).

The clamp 100 may be positioned so that the left atrium 816 (see FIG. 24) is clamped between the jaws 502, 504, without the jaws 502, 504 engaging the right atrium. The tips 514, 516 of the jaws 502, 504 may be positioned generally anterior to the left pulmonary veins 814. The jaws 502, 504 may be generally posterior to the left atrial appendage 818. Upon reaching the position shown in FIG. 27, the controller and RF generator 14, which are communicatively coupled to the clamp 100 and individually communicatively coupled to each segmented electrode pair (so the controller knows the relative position of each segmented electrode pair of the clamp), activates a sequence where each of the segmented electrode pairs (528, 532), (530, 534), (535, 539), (537, 541), (543, 547), (545, 549), (551, 555), (553, 557), (559, 563), (561, 565), (567, 571), (569, 573), (575, 579), (577, 581) (see FIGS. 17, 18) is energized with non-ablative energy so that readings from the segmented electrode pairs allow the controller 14 to calculate the impedance of tissue interposing each segmented electrode pair. As discussed, if the calculated tissue impedance for a given segmented electrode pair is less than a predetermined threshold, then the controller 14 will deliver energy to that given segmented electrode pair individually. Conversely, if the calculated tissue impedance for that given segmented electrode pair is equal to or more than a predetermined threshold, then the controller 14 looks to the next adjacent segmented electrode pair and uses an impedance average across these two segmented electrode pairs. If the impedance average is less than the predetermined threshold, then the controller 14 will group the two segmented electrode pairs as a single unit and distribute energy that will be equally divided amongst the segmented electrode pairs when the ablation sequence commences. In this manner, the two segmented electrode pairs would be grouped and operated as such, which effectively inhibits operating either of these two segmented electrode pairs individually. Conversely, if the impedance average is equal to or more than the predetermined threshold, then the controller 14 will move on to the next adjacent segmented electrode pair and average the impedance calculations across three pairs of segmented electrodes. This process continues until the average impedance is below the predetermined threshold or there are no more pairs of segmented electrodes available. In the former case, the necessary segmented electrode pairs are grouped as a single unit and operated as such, followed by the controller 14 moving on to the next adjacent segmented electrode pair to discern whether the next segmented electrode pair can be operated individually or must be combined/grouped with another segmented electrode pair and operated as a unit. In the latter case, where there are no other segmented electrode pairs for further impedance averages, the controller 14 will end the sequence by grouping all segmented electrode pairs contributing to the impedance average most recently calculated and operate these segmented electrode pairs grouped as a single unit. What this means is that power supplied by the RF generator is equally divided across each of the segmented electrode pairs within that group.

After determining which of the segmented electrode pairs (528, 532), (530, 534), (535, 539), (537, 541), (543, 547), (545, 549), (551, 555), (553, 557), (559, 563), (561, 565), (567, 571), (569, 573), (575, 579), (577, 581) are to be driven individually or in parallel (via the grouping), the controller 14 is ready to begin the ablation sequence. As part of determining which segmented electrode pairs will be driven individually and those that will be driven as a group, the controller 14 includes a visual display 600 that may display an initial indication as shown in FIG. 27A. This visual display includes a rounded rectangle for each of the fourteen segmented electrode pairs that are part of the clamp 100. Each of these rounded rectangles is colored red, signifying that the ablation has yet to commence and that none of the segmented ablation electrodes are being powered with ablation energy. Also, the visual display 600 includes two brackets, which denote to the user that the third, fourth, and fifth segmented electrode pairs are being operated as a first group, while the tenth and eleventh segmented electrode pairs are being operated as a second group, while all other segmented electrode pairs are being operated individually.

In exemplary form, a user of the clamp 100 will interface with the controller 14 to initiate the ablation sequence. At some time after the ablation sequence is initiated by the user, the visual display 600 (see FIG. 21) may change to that shown in FIG. 27B. As part of this display, the rounded rectangles corresponding to the individually driven segmented electrode pairs (1^{st}, 2^{nd}, 6^{th}-9^{th}, 12^{th}-14^{th}) have changed in color and are colored yellow, which in exemplary form conveys the ablation process has surpassed a first threshold, but not yet achieved a second threshold. Conversely, the rounded rectangles corresponding to the grouped segmented electrode pairs (3^{rd}-5^{th}, 10^{th}, 11^{th}) have changed in color and are colored orange, which in exemplary form conveys the ablation process has been initiated but the tissue ablation has not yet met a first threshold. The controller 14 is able to determine the percent completion of the ablation or progress of the ablation by periodically calculating the impedance of the tissue interposing the segmented electrode pairs.

At some later time, after the time reflected by the display 600 of FIG. 27B, the display may be revised to depict the images shown in FIG. 27C. As part of this display, the rounded rectangles corresponding to the individually driven segmented electrode pairs (1^{st}, 2^{nd}, 6^{th}-9^{th}, 12^{th}-14^{th}) have changed in color and are colored light green, which in exemplary form conveys the ablation process has surpassed a second threshold, but not yet achieved completion of the ablation sequence. Conversely, the rounded rectangles corresponding to the grouped segmented electrode pairs (3^{rd}-5^{th}, 10^{th}, 11^{th}) have changed in color and are colored yellow, which in exemplary form conveys the ablation process has surpassed a first threshold, but not yet achieved a second threshold. It should be noted that the display continues to reflect that the rounded rectangles corresponding to the grouped segmented electrode pairs (3^{rd}-5^{th}, 10^{th}, 11^{th}) continue to be grouped and operated as two units rather than five individual segmented electrode pairs.

At some even later time, after the time reflected by the display 600 of FIG. 27C, the display may be revised to depict the images shown in FIG. 27D. As part of this display, the rounded rectangles corresponding to the individually driven segmented electrode pairs (1^{st}, 2^{nd}, 6^{th}-9^{th}, 12^{th}-14^{th}) have changed in color and are colored dark green, which in exemplary form conveys the ablation process has been completed and the segmented ablation electrode pairs are no longer receiving ablative energy. Conversely, the rounded rectangles corresponding to the grouped segmented electrode pairs (3^{rd}-5^{th}, 10^{th}, 11^{th}) have changed in color and are colored light green, which in exemplary form conveys the ablation process has surpassed a second threshold, but not yet achieved completion of the ablation sequence. It should be noted that the display continues to reflect that the rounded rectangles corresponding to the grouped segmented electrode pairs (3^{rd}-5^{th}, 10^{th}, 11^{th}) continue to be grouped and operated as two units rather than five individual segmented electrode pairs.

At a still later time, after the time reflected by the display 600 of FIG. 27D, the display may be revised to depict the images shown in FIG. 27E. As part of this display, the rounded rectangles corresponding to the individually driven segmented electrode pairs (1^{st}, 2^{nd}, 6^{th}-9^{th}, 12^{th}-14^{th}) remain colored dark green, which confirms the ablation process is complete and these segmented ablation electrode pairs are no longer receiving ablative energy. Similarly, the rounded rectangles corresponding to the grouped segmented electrode pairs (3^{rd}-5^{th}, 10^{th}, 11^{th}) have changed in color and are colored dark green, which in exemplary form conveys the ablation process is complete and these segmented ablation electrode pairs are no longer receiving ablative energy. It should also be noted that the display no longer displays brackets to reflect that the rounded rectangles previously corresponding to the grouped segmented electrode pairs (3^{rd}-5^{th}, 10^{th}, 11^{th}) are no longer grouped together. Upon the user visually verifying the ablation sequence is complete, the user may open and reposition the clamp 100 elsewhere on the patient's tissue and follow a similar process once the tissue to be ablated is clamped by the clamp. Upon the user determining that no more tissue is to be ablated using the clamp 100, the clamp may be withdrawn from the patient's body and the one or more incisions closed.

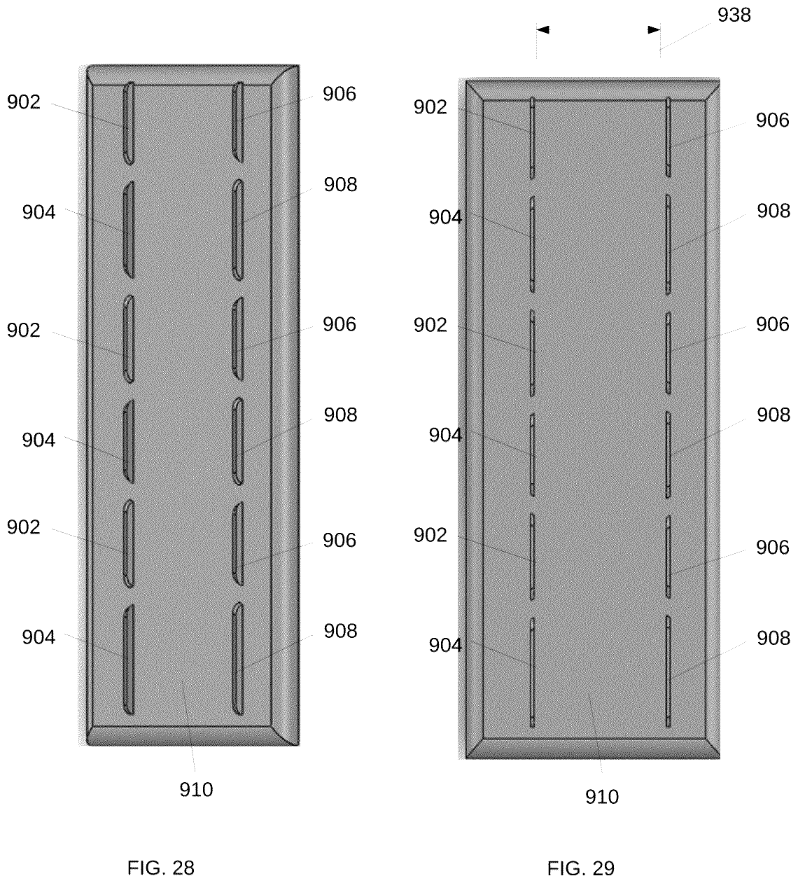

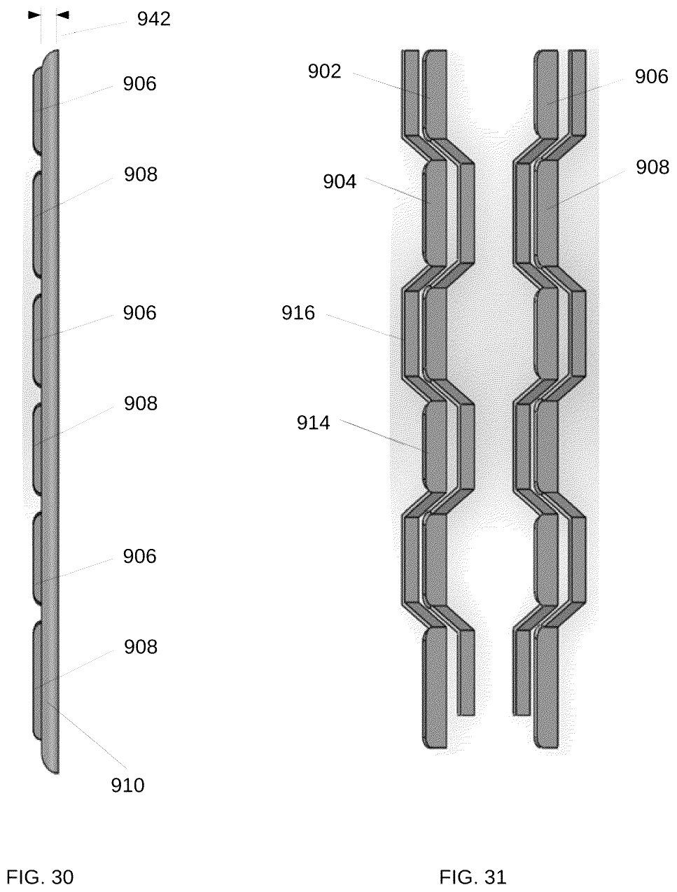

Turning to FIGS. 28-33, it is also within the scope of the disclosure to provide an electrosurgical instrument with segmented electrodes 902-908, where two or more of the electrodes extend above an insulating layer 910 in discrete segments that are actually an integrated electrode.

By way of example, an exemplary insulated substrate 910 may include two or more electrode rows, with each row including a plurality of discrete, exposed electrode contacts configured to contact tissue to be ablated. Each row may be linear, may be arcuate, or may take on other shapes, such as those known or expected by one skilled in the art. In exemplary form, the electrodes of each row may be linearly aligned or regularly offset from one another in the widthwise direction, akin to a staggered or alternating staggered pattern. In any event, presuming each row includes six electrode contacts distributed longitudinally, the first, third, and fifth electrode contacts exposed are really part of the same first electrode 902, 906. Similarly, the second, fourth, and sixth electrode contacts exposed are really the same second electrode 904, 908. It is to be understood that fewer than six and more than six electrode contacts may be provided as part of each row, with a similar division being imparted by more or less than six electrode contacts per row.

By providing distinct electrode contacts, in lieu of a continuous electrode length (where the continuous electrode length comprising 50% of electrode 902, and the other 50% comprising electrode 904), the current may be distributed more evenly along the length of each electrode. By breaking up a single continuous electrode into a plurality of distinct electrode contacts, this structure may be more effective at distributing energy more evenly across the tissue contacted.

In exemplary form, the segmented electrodes 902-908 and insulating layer 910 may be part of an electrosurgical instrument, such as an electrosurgical clamp as disclosed herein. More specifically, the segmented electrodes 902-908 and insulating layer 910 may be part of one or more jaws of an electrosurgical clamp as disclosed herein.

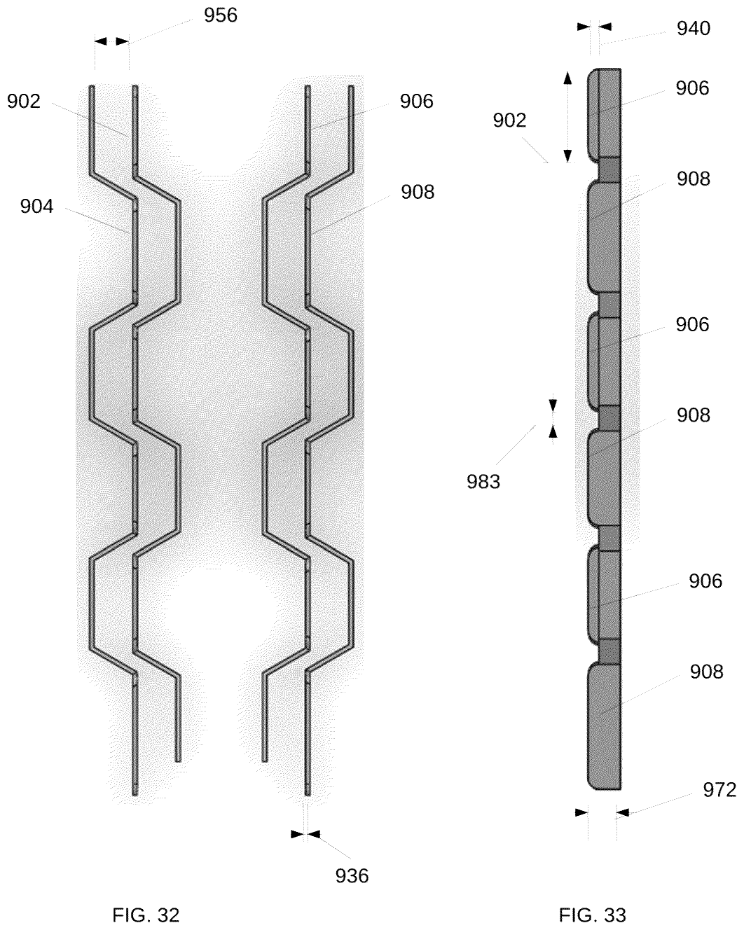

In exemplary form, the each of the electrodes 902-908 may include complementary segments that are in parallel to one another. By way of example, a plurality of precursor electrode strips may be punched from a sheet material to have a longitudinal profile with raised electrode segments 914 interposed by lower contact portions 916. These precursor electrode strips may then be loaded into one of two undulating presses, with a first batch longitudinally offset from a second batch in each press. Each of the first batch, from the first press, creates a first electrode 902 (see FIG. 32), while each of the second batch, from the first press, creates a second electrode 904. Similarly, each of the first batch, from the second press, creates a third electrode 906, while each of the second batch, from the second press, creates a fourth electrode 908. The first electrode 902 includes six linear segments that are interconnected via alternating angled segments at 135 degrees and 225 degrees in order to orient the second, fourth, and sixth of the linear segments (raised electrode segments 914) along a longitudinal straight line. The second electrode 904 includes six linear segments that are interconnected via alternating angled segments at 135 degrees and 225 degrees in order to orient the first, third, and fifth of the linear segments (raised electrode segments 914) along a longitudinal straight line. Similar structures, that are mirror images of electrodes 902, 904 are accomplished for electrodes 906, 908 via the second press.

The pressed electrodes 902-908 may be orientated as shown in FIG. 32, so that the raised electrode segments 914 are longitudinally aligned, and placed in mold to be injected with insulating material 910 to ensure the electrodes are electrically isolated from one another, which results in a jaw intermediary as depicted in FIGS. 28-30. In this manner, what appears to be a linear row of a series of electrode contacts may comprise a fewer number of electrodes that need not be independently driven. For example, in the context of a six electrode contact row, it may be presumed that each electrode contact requires its own individual electrical lead, which would in such a case require six leads. But by staggering the two electrodes 902, 904 (and 906, 908), one is able to provide a linear segment (or other shape) of multiple electrode contacts, where the number of required leads to drive the electrode contacts is less than the number of electrode contacts (in this case, two electrical leads, one for electrode 902, a second for electrode 904). This jaw intermediary may be mounted to a jaw of an electrosurgical instrument, as disclosed herein, and may include separate leads to each of the electrodes 902-908.

In exemplary form, an exemplary electrosurgical device may include four or more pairs of opposed segmented electrodes 902-908 as referenced herein. In exemplary form, where the electrosurgical device comprises a clamp, a first jaw may include the segmented electrodes 902-908 seated within the insulating layer 910 that encases the segmented electrodes. Similarly, a second jaw may include a corresponding segmented electrodes 902-908 also seated within an insulating layer 910. Though not required, the electrodes 902-908 may extend above the insulating layer and start and/or terminate at different longitudinal locations along each jaw. Conversely, the electrodes 902-908 may have extend above the insulating layer 910 and have terminal ends that approximate one another in a longitudinal direction. In exemplary form, the electrodes 902-908 may be centered laterally on each jaw and spaced apart a distance of from about 0.7 mm to about 4.0 mm. It should be noted, however, that electrode spacings smaller than 0.7 mm and larger than 4.0 mm may be utilized. These dimensions are illustrative only, and other dimensions may be used without departing from the present disclosure.

Each electrode of the segmented electrode pairs may have a width 936, which may be the width of the tissue-facing surface in a direction generally perpendicular to the local, elongated direction of the electrode. Moreover, each electrode may be spaced apart by an electrode spacing 938. Each electrode may extend beyond the surface of the insulator 910 by a projection height 940. Each electrode may be spaced at an insulation depth 942 from its respective jaw beam. Each electrode may have a total electrode height 972.

In some example embodiments, the electrode width 936 of the segmented electrode pairs may be about 0.1 mm to about 2.0 mm. In some example embodiments, the electrode width 936 may be about 0.2 to about 0.4 mm. In some example embodiments, the electrode width 936 may be about 0.30 mm. In some example embodiments, each electrode may have substantially the same width 936, which may be substantially constant over the length each electrode. In other embodiments, the width 936 may vary over the length of an electrode and/or may differ from electrode to electrode.

In some example embodiments, the widthwise electrode spacing 938 of the segmented electrode pairs may be about 0.1 mm to about 3.0 mm. In some example embodiments, the widthwise electrode spacing 938 may be about 0.3 mm to about 0.6 mm. In some example embodiments, the widthwise electrode spacing 938 may be about 0.43 mm. In some example embodiments, the widthwise electrode spacing 938 may be substantially constant over the length of the plurality of electrodes so that the electrodes are substantially parallel in any local region. In other embodiments, the widthwise electrode spacing 938 may vary over the length of the plurality of electrodes so that the electrodes may be closer together in some regions and/or farther apart in other regions. Generally, reducing the electrode spacing 938 may result in narrower lesions and/or faster ablation.

In some example embodiments, the electrode widthwise spacing 956 between staggered electrodes 902, 904 and 906, 908 may be about 1.0 mm to about 20 mm. In some example embodiments, the electrode widthwise spacing 956 may be about 2.0 to about 4.0 mm. In some example embodiments, the electrode widthwise spacing 956 may be about 3.0 mm. In some example embodiments, widthwise spacing 956 between complementary electrodes may be substantially constant over the length each electrode, or the spacing may vary over the length of the electrodes and/or may differ from electrode to electrode.

In some example embodiments, the longitudinal electrode spacing 983 of the segmented electrode pairs may be about 0.1 mm to about 3.0 mm. In some example embodiments, the longitudinal electrode spacing 983 may be about 0.3 mm to about 0.6 mm. In some example embodiments, the longitudinal electrode spacing 983 may be about 0.43 mm. In some example embodiments, the longitudinal electrode spacing 983 may be substantially constant over the total length of the plurality of electrodes. In other embodiments, the longitudinal electrode spacing 983 may vary over the total length of the plurality of electrodes so that some electrodes may be closer together in some regions and/or farther apart in other regions.

In some example embodiments, the projection height 940 may be about 0.0 mm (e.g., flush) to about 0.5 mm. In some example embodiments, the projection height 940 may be about 0.1 mm to about 0.2 mm. In some example embodiments, the projection height 940 may be about 0.15 mm. In some example embodiments, the projection height 940 may be substantially constant over the length of a particular electrode and/or may be substantially the same for two or more electrodes. In other embodiments, the projection height 940 may vary over the length of an electrode and/or from electrode to electrode.

In some example embodiments, the insulation depth 942 may be about 0.1 mm to about 5.0 mm. In some example embodiments, the insulation depth 942 may be about 0.8 mm to about 1.6 mm. In some example embodiments, the insulation depth 942 may be about 1.25 mm. In some example embodiments, the insulation depth 942 may be substantially constant over the length of a particular electrode and/or may be substantially the same for two or more electrodes. In other embodiments, the insulation depth 942 may vary over the length of an electrode and/or from electrode to electrode. Generally, increasing the insulation depth 942 may result in narrower lesions, faster ablation, and/or lower energy per unit volume.

In some example embodiments, the electrode height 972 may be about 0.25 mm to about 3.0 mm. In some example embodiments, the electrode height 972 may be about 0.3 mm to about 0.7 mm. In the example embodiments, the electrode height 972 may be about 0.5 mm. In some example embodiments, the electrode height 972 may be substantially constant over the length of a particular electrode and/or may be substantially the same for two or more electrodes. In other embodiments, the electrode height 972 may vary over the length of an electrode and/or from electrode to electrode.

In some example embodiments, the electrodes of the segmented electrode pairs may extend substantially the entire length of the jaws. By way of example, the electrodes of the segmented electrode pairs on each jaw may have a total length of about 105 mm. In an alternative exemplary embodiment with shorter jaws, the electrodes of the segmented electrode pairs may have a total length of about 86 mm.

In some example embodiments according to at least some aspects of the present disclosure, the jaws may be configured to facilitate positioning adjacent to and engagement of a particular target tissue in a desired manner. For example, the shape of the jaws may be selected based on the target tissue and/or the location of the target tissue in relation other anatomical structures. As described in more detail hereafter, the jaws may be used to create lesions around the pulmonary veins, which are located generally on the posterior portion of the heart. When the heart is accessed via a median sternotomy, creating lesions around the pulmonary veins may require positioning the jaws at least partially around the posterior aspect of the heart, while engaging the left atrium and avoiding nearby structures that will not be ablated.

In some example embodiments, the jaws may be linear or substantially linear, or formed of one or more substantially straight (e.g., generally linear) portions, which may be interposed by one or more generally curved or bent portions. For example, in the first jaw, a first substantially straight portion may extend from the first end portion to a first curved portion. A second substantially straight portion may extend from the first curved portion to a second curved portion. A third substantially straight portion may extend from the second curved portion to the second end portion. In some example embodiments, each of the substantially straight portions may be obliquely oriented (e.g., non-parallel and non-perpendicular) with respect to each of the other substantially straight portions. For example, an angle between the first substantially straight portion and the second substantially straight portion may be about 110 degrees to about 150 degrees and/or an angle between the second substantially straight portion and the third substantially straight portion may be about 110 degrees to about 150 degrees. By way of example, a first substantially straight portion may have a length of about 2.9 cm, a second substantially straight portion may have a length of about 5.0 cm, and a third substantially straight portion may have a length of about 2.8 cm, with angles therebetween being between about 100 degrees to about 165 degrees.

By way of example, in furtherance of carrying out an ablation, RF energy may be delivered from control system/RF generator 14 individually to each of a plurality of segmented electrode pairs 902-908 or may be delivered and divided across two or more segmented electrode pairs. Unless otherwise noted, it is to be understood that if RF energy is delivered to a segmented electrode pair, this RF energy is not diminished, degraded, or otherwise eroded regardless of the number of segmented electrode pairs concurrently active.

In exemplary form, prior to carrying out an active tissue ablation, the control system/RF generator 14 may utilize one or more of the segmented electrode pairs, when tissue is clamped therebetween, to discern what the impedance of the clamped tissue is at predetermined intervals. By way of example, these predetermined intervals may correspond to the intervals of the locations of each of the segmented electrode pairs. Determining the impedance of clamped tissue may allow the control system/RF generator 14 to determine an RF energy for each segmented electrode pair that may be location specific. Put another way, by determining the tissue impedance at each of the segmented electrode pairs, the control system/RF generator 14 is able determine how much RF energy is delivered to each segmented electrode pair and whether two or more of the segmented electrode pairs will be effectively operated in parallel (dividing a singular RF energy across two or more segmented electrode pairs).

By way of example, the control system/RF generator 14 may initially direct alternating current through a first of the electrodes of each of the segmented electrode pairs when tissue is clamped between the jaws. This alternating current flows into the tissue and the resulting voltage of this alternating current is measured by the second of the electrodes of each of the segmented electrode pairs. The resulting voltage drop across the tissue is measured and the control system/RF generator 14 calculates the impedance using Ohm's law by combining resistance and reactance. Based upon programming within the control system/RF generator 14, a predetermined impedance threshold determines whether the segmented electrodes will be operated individually or in parallel (RF energy divided across two or more segmented electrodes). By way of example, if the determined impedance across a segmented electrode pair is less than the predetermined impedance threshold (such as, without limitation, 450 ohms), then that segmented electrode pair may be operated individually, meaning that RF power supplied to that segmented electrode pair will not be distributed to another segmented electrode pair. While a first segmented electrode pair may be driven at the same RF power rate as a second segmented electrode pair, the total RF power delivered to the first segmented electrode pair is not dependent upon another segmented electrode pair. For those segmented electrode pairs having an impedance below the predetermined impedance threshold, each segmented electrode pair may be driven individually and continuously by the control system/RF generator 14 until the measured impedance during the ablation across the electrodes of that segmented electrode pair meets or exceeds an ablation impedance threshold. Upon reaching or exceeding the ablation impedance threshold across a segmented electrode pair driven individually, the control system/RF generator 14 may continue RF power to that segmented electrode pair.

Alternatively, when the control system/RF generator 14 determines the impedance of tissue 102 across a segmented electrode pair meets or exceeds a predetermined impedance threshold, then the control system/RF generator 14 may distribute RF power concurrently across two or more segmented electrode pairs and operate these segmented electrodes as a single unit. After determining whether each segmented electrode pair will be operated independently or operated in conjunction with one or more segmented electrode pairs, the control system/RF generator 14 may initialize the ablation sequence and power the segmented electrode pairs. The powering of all or fewer than all of the segmented electrode pairs may be accomplished simultaneously. In this fashion, the control system/RF generator 14 actively monitors the segmented electrode pairs to which RF power is supplied and continues the ablation across the segmented electrode pairs until reaching a predetermined ablation completion metric. By way of example, adjacent segmented electrode pairs may be alternatively activated and deactivated to originate a tissue lesion under the surface of the exposed tissue to be ablated.

Following from the above description, it should be apparent to those of ordinary skill in the art that, while the methods and apparatuses herein described constitute exemplary embodiments of the present invention, the invention described herein is not limited to any precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

What is claimed is:

1. An ablation system comprising:
a controller;
an energy generator operatively coupled to the controller; and
a surgical device including a plurality of segmented electrode pairs, the plurality of segmented electrode pairs being operatively coupled to the controller but electrically isolated from one another;
wherein the controller is configured to individually power at least a portion of or all of the plurality of segmented electrode pairs and/or to power at least a portion of or all of the plurality of segmented electrode pairs as a group.

2. The ablation system of claim 1, wherein:
the surgical device comprises a surgical clamp that includes a first jaw and a second jaw, where at least one of the first jaw and the second jaw is repositionable; and
the plurality of segmented electrode pairs are distributed across the first jaw and the second jaw.

3. The ablation system of claim 1, wherein:
the plurality of segmented electrode pairs includes a first segmented electrode pair, a second segmented electrode pair, a third segmented electrode pair, and a fourth segmented electrode pair;
the first jaw includes a first electrode of each of the first segmented electrode pair, the second segmented electrode pair, the third segmented electrode pair, and the fourth segmented electrode pair; and
the second jaw includes a first electrode of each of the first segmented electrode pair, the second segmented electrode pair, the third segmented electrode pair, and the fourth segmented electrode pair.

4. The ablation system of claim 1, wherein:
the controller includes a visual display including visual indicium representing each of the plurality of segmented electrode pairs; and
the visual display is updated to reflect progress of an ablation sequence using the plurality of segmented electrode pairs.

5. The ablation system of claim 4, wherein the visual indicium includes numerical representations regarding the progress of the ablation sequence.

6. The ablation system of claim 4, wherein the visual indicium includes colored representations regarding the progress of the ablation sequence.

7. The ablation system of claim 6, wherein the colored representations change as a function of nearing completion of the ablation sequence.

8. The ablation system of claim 6, wherein the colored representations include a first shape denoting a first of the plurality of segmented electrode pairs is being powered individually, and a second shape denoting at least a second of the plurality of segmented electrodes is not being powered individually.

9. The ablation system of claim 1, wherein:
the surgical device includes a first jaw and a second jaw, where at least one of the first jaw and the second jaw is repositionable;
the plurality of segmented electrode pairs includes a first segmented electrode pair, a second segmented electrode pair, a third segmented electrode pair, and a fourth segmented electrode pair;
the first jaw includes a first electrode of each of the first segmented electrode pair, the second segmented electrode pair, the third segmented electrode pair, and the fourth segmented electrode pair;
the second jaw includes a first electrode of each of the first segmented electrode pair, the second segmented electrode pair, the third segmented electrode pair, and the fourth segmented electrode pair; and
at least two of the electrodes of the first jaw are oriented in parallel to one another.

10. The ablation system of claim 9, wherein:
a first of the electrodes of the first jaw is oriented parallel to second and third electrodes of the first jaw; and
the first electrode overlaps both the second and third electrodes in a direction normal to the parallel orientation.

11. A method of carrying out an ablation process using a plurality of segmented electrode pairs, the method comprising:
sandwiching tissue between a first segmented electrode pair, a second segmented electrode pair, a third segmented electrode pair, and a fourth segmented electrode pair, where a first electrode of each segmented electrode pair is on a first side of the tissue, and where a second electrode of each segmented electrode pair is on a second, opposite side of the tissue; and
individually and concurrently powering each of the first segmented electrode pair, the second segmented electrode pair, the third segmented electrode pair, and the fourth segmented electrode pair without regard to power delivered to the other segmented electrode pairs.

12. The method of claim 11, further comprising:
determining impedance of the tissue sandwiched between the first segmented electrode pair, the second segmented electrode pair, the third segmented electrode pair, and the fourth segmented electrode pair to generate four separate tissue impedances;
wherein each of the four separate tissue impedances are below a first threshold impedance.

13. The method of claim 12, further comprising:
sandwiching tissue between a fifth segmented electrode pair and a sixth electrode pair, where a first electrode of the fifth and sixth electrode pairs is on the first side of the tissue, and where a second electrode of the fifth and sixth electrode pairs is on the second, opposite side of the tissue; and
concurrently powering each of the fifth segmented electrode pair and the sixth segmented electrode pair by equally distributing an ablation energy from an energy generator.

14. The method of claim 13, further comprising:
determining impedance of the tissue sandwiched between the fifth segmented electrode pair and the sixth segmented electrode pair, to generate fifth and sixth separate tissue impedances;
wherein at least one of the fifth and sixth separate tissue impedances is above a first threshold impedance;
wherein the fifth and sixth segmented electrode pairs are operated as a single unit.

15. The method of claim 14, further comprising:
determining impedance of the tissue sandwiched between the fifth segmented electrode pair and the sixth segmented electrode pair after concurrently powering the fifth and sixth segmented electrode pairs; and
discontinuing power to both the fifth and sixth segmented electrode pairs when the determined tissue impedance is above a threshold ablation metric.

16. The method of claim 11, further comprising:
determining impedance of the tissue sandwiched between the first segmented electrode pair, the second segmented electrode pair, the third segmented electrode pair, and the fourth segmented electrode pair to generate four separate tissue impedances after concurrently powering the first, second, third, and fourth segmented electrode pairs;
wherein at least one of four separate tissue impedances corresponding to the first segmented electrode pair is above a threshold ablation metric; and
revising the individually and currently powering the first and second segmented electrode pairs to power the first and second segmented electrode pairs as a single group so the same power is distributed equally across the first and second segmented electrode pairs.

17. The method of claim 11, further comprising:
determining impedance of the tissue sandwiched between the first segmented electrode pair, the second segmented electrode pair, the third segmented electrode pair, and the fourth segmented electrode pair to generate four separate tissue impedances after concurrently powering the first, second, third, and fourth segmented electrode pairs;
wherein at least one of four separate tissue impedances is above a threshold ablation metric; and
revising the individually and concurrently powering of those of the first, second, third, and fourth segmented electrode pairs when a tissue impedance across one or more of the first, second, third, and fourth segmented electrode pairs is above the threshold ablation metric and, instead, group powering at least two of the first, second, third, and fourth segmented electrode pairs.

18-20. (canceled)

21. An ablation system comprising:
an energy generator; and
a surgical device including a plurality of segmented electrode pairs, the plurality of segmented electrode pairs being electrically isolated from one another but operatively coupled to the energy generator;
wherein the energy generator is configured to individually power at least a portion of or all of the plurality of segmented electrode pairs and/or to power all of or at least a portion of the plurality of segmented electrode pairs as a group dependent upon tissue impedance.

22-30. (canceled)

31. An electrosurgical instrument, comprising:
an ablation structure including a first electrode and a second electrode;
wherein the first electrode includes a plurality of first electrode contacts;
wherein the second electrode includes a plurality of second electrode contacts;
wherein the plurality of first electrode contacts and the plurality of second electrode contacts are configured to be available to contact tissue;
wherein the plurality of first electrode contacts is interconnected by a first conductor buried in a first insulating material;
wherein the plurality of second electrode contacts is interconnected by a second conductor buried in a first insulating material;
wherein the plurality of first electrode contacts and the plurality of second electrode contacts lie in a first row.

32-51. (canceled)
